SEQUENCE LISTING (12 sequences)

SEQ ID NO: 1 - protein, 1037 aa, Artificial Sequence
  AlphaIIb monomeric subunit
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERGGGSGGGSGGGSDYKDDDDKGDYKDDDDKIDYKDDDDKHHHHHH

SEQ ID NO: 2 - protein, 993 aa, Artificial Sequence
  Alpha IIb moiety
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEER

SEQ ID NO: 3 - protein, 12 aa, Artificial Sequence
  Linker for AlphaIIb
GGGSGGGSGGGS

SEQ ID NO: 4 - protein, 26 aa, Artificial Sequence
  Acidic tail for AlphaIIb
DYKDDDDKGDYKDDDDKIDYKDDDDK

SEQ ID NO: 5 - protein, 6 aa, Artificial Sequence
  Histidine tag for AlphaIIb
HHHHHH

SEQ ID NO: 6 - protein, 744 aa, Artificial Sequence
  Beta3 monomeric subunit
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDHPGGGSGGGSGGGSWKKYKKSDPGWKKYKKSDGGWKKYKKSDPGWKKYKWSD

SEQ ID NO: 7 - protein, 692 aa, Artificial Sequence
  Beta3 moiety
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPD

SEQ ID NO: 8 - protein, 14 aa, Artificial Sequence
  Linker for Beta3
HPGGGSGGGSGGGS

SEQ ID NO: 9 - protein, 79 aa, Artificial Sequence
  Basic tail for Beta3
RFQYYEDSSGKSILYVVEEPECPKGPDHPGGGSGGGSGGGSWKKYKKSDPGWKKYKKSDGGWKKYKKSDPGWKKYKWSD

SEQ ID NO: 10 - protein, 500 aa, Artificial Sequence
  GPIbalpha chimeric unit
HPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRATRTVVKFPTKAHTTPWGLFYSWSTASLDSQMPSSLHPTQESTKEQTTFPPRWTPNFTLHMESITFSKTPKSTTEPTPSPTTSEPVPEPAPNMTTLEPTPSPTTPEPTSEPAPSPTTPEPTSEPAPSPTTPEPTSEPAPSPTTPEPTPIPTIATSPTILVSATSLITPKSTFLTTTKPVSLLESTKKTIPELDQPPKLGPDYKDDDDK

SEQ ID NO: 11 - protein, 490 aa, Artificial Sequence
  GPIbalpha moiety
HPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRATRTVVKFPTKAHTTPWGLFYSWSTASLDSQMPSSLHPTQESTKEQTTFPPRWTPNFTLHMESITFSKTPKSTTEPTPSPTTSEPVPEPAPNMTTLEPTPSPTTPEPTSEPAPSPTTPEPTSEPAPSPTTPEPTSEPAPSPTTPEPTPIPTIATSPTILVSATSLITPKSTFLTTTKPVSLLESTKKTIPELDQPPKL

SEQ ID NO: 12 - protein, 8 aa, Artificial Sequence
  Flat tag for GPlbalpha
DYKDDDDK

(19) United States
(12) Patent Application Publication
(10) Pub. No.: US 2023/0194519 A1
(43) Pub. Date: Jun. 22, 2023

(54) RAPID AND FACILE ANTIBODY DETECTION USING COVALENTLY IMMOBILIZED SELF-ASSEMBLED POLYPEPTIDES

(71) Applicant: CCOA THERAPEUTICS INC., Toronto (CA)

(72) Inventors: Heyu NI, North York (CA); Miguel NEVES, São Vítor, Braga (PT)

(21) Appl. No.: 17/997,182

(22) PCT Filed: Apr. 27, 2021
(86) PCT No.: PCT/CA2021/050574

§ 371 Date: Oct. 26, 2022

Related U.S. Application Data
(60) Provisional application No. 63/015,810, filed on Apr. 27, 2020.

(51) Int. Cl. G01N 33/564; C07K 17/14

(52) U.S. Cl. CPC G01N 33/564; C07K 17/14; C07K 2319/735; G01N 2800/222

(57) ABSTRACT

Methods are provided for determining the presence of antibodies in blood or a blood product, using immobilized self-assembled polypeptides comprising an ectodomain and being recognized by the antibodies. The self-assembled polypeptide comprises at least a first chimeric polypeptide. In the methods the functionality and active conformation of the immobilized and self-assembled polypeptides is preserved. Processes for making the immobilized self-assembled polypeptides are also provided.

### TECHNOLOGICAL FIELD

The present disclosure relates to methods and surfaces for detecting antibodies in plasma using immobilized polypeptides that are recognized by the antibodies.

### BACKGROUND

Thrombocytopenia is a condition associated with a low blood platelet count. Immune thrombocytopenia (ITP) is an immune bleeding disorder that leads to low platelet counts and the risk of death. Although the exact cause of ITP remains poorly understood, the literature shows that the vast majority of ITP cases are mediated by autoantibodies produced against the platelet surface receptors GPIbα and/or αIIbβ3 (GPIIbIIIa). Recently, it was shown that the platelet clearance mechanisms of GPIbα- and αIIbβ3-mediated ITP are different and do not respond to the same types of treatment. Antibody-mediated platelet clearance in ITP can be divided into two pathways: 1) Fc-dependent: antibody binds the platelet via the Fab portion, bridging the opsonized platelet to macrophage Fc receptors. Opsonized platelets are subsequently engulfed and cleared by macrophages (McMillan et al., 1974). However, the above Fc-dependent models cannot explain all mechanisms of platelet clearance, as Fc-removed anti-GPIbα monoclonal antibodies (mAbs) still induce thrombocytopenia (Nieswandt et al., 2000). 2) Fc-independent: this novel pathway is currently yet to be fully characterized but has been observed (Li et al., 2015; Webster et al., 2006) (Nieswandt et al., 2000). Recent findings demonstrate that most anti-GPIbα induced platelet desialylation and AMR-related hepatic platelet clearance have significant implications for both diagnosis and therapy (Li et al., 2015; Xu et al., 2018). Further understanding of this novel Fc-independent pathway should be of great significance in the clinical management of ITP, since the first line treatments that are effective against anti-αIIbβ3 mediated ITP such as, intravenous immunoglobulin (IVIG), steroids, anti-D, and splenectomy are often found to be ineffective in patients with these activating anti-platelet (anti-GPIbα) antibodies (Li et al., 2015; Xu et al., 2018; Tao et al., 2017; Peng et al., 2014). In patients with activating anti-platelet (anti-GPIbα) antibodies, sialidase treatment has provided some therapeutic benefits.³ Therefore, a clinical detection assay would be beneficial to assist physicians in the diagnosis and treatment of ITP.

Anti-platelet autoantibodies in ITP patients are of low affinity and concentrations, detection is difficult with possible false negatives due to the low sensitivity of traditional methods including ELISA and flow cytometry. The current gold standard of ITP autoantibody detection, termed Monoclonal Antibody Immobilization of Platelet Antigens or MAIPA, is rarely clinically utilized as it is time consuming (2-3 days), requires a large amount of patient blood sample (usually 90-120 mL), is very labor intensive, lacks sensitivity (e.g., only ˜60% of ITP patients have MAIPA-detectable autoantibody) often leading tofalse negative results and can be prone to false positive results (˜30%) when murine antibodies are utilized without extra washing steps. (Curtis & McFarland, 2009; Klee, 2000; Metzner et al., 2017)

It would be highly desirable to be provided with reagents capable of detecting (auto-) antibodies in the blood or a blood product which have increased specificity and/or do not require complex laboratory procedures.

### BRIEF SUMMARY

The present disclosure concerns the use of a self-assembled polypeptide (comprising an ectodomain) immobilized on a surface for the detection of antibody present in blood or a blood product.

In a first aspect, the present disclosure concerns a method of determining the presence of an antibody specific for a polypeptide present in blood or a blood product. The method comprises: a) contacting (i) at least one first self-assembled polypeptide immobilized on a surface, the at least one first self-assembled polypeptide comprising a first ectodomain moiety, with (ii) a sample suspected of comprising the antibody; and b) detecting the presence or absence of a complex between the at least one of the first self-assembled polypeptide and the antibody, wherein the presence of the complex is indicative of the presence of the antibody in the sample. The at least one first self-assembled polypeptide comprises a first chimeric polypeptide of formula (Ia) or (Ib):

NH₂-FPM-FAAL-FAT-COOH (Ia)

NH₂-FAT-FAAL-FPM-COOH (Ib)

wherein FPM is a first polypeptide moiety derived from the polypeptide present in the blood or the blood product; FAAL is a first optional amino acid linker; FAT is a first amino acid tail having at least one acidic amino acid residue each having an R-group comprising a carboxyl group; — is an amine bond; the carboxyl group of the first chimeric polypeptide is covalently associated to a first silane linker (FSL) moiety, wherein the FSL is covalently associated with at least one first hydroxyl group of the surface; and the at least one self-assembled polypeptide has specific affinity to the antibody. In an embodiment, the sample is from a subject suspected of comprising the antibody. In another embodiment, the blood product is a plasma. In still a further embodiment, the plasma is a platelet-rich plasma or a platelet-poor plasma. In some embodiments, the method is for diagnosing thrombocytopenia, wherein the detection of the complex is indicative of the presence of thrombocytopenia in the subject. In such embodiment, the polypeptide can be a polypeptide present on the surface of a platelet. In a specific embodiment, the antibody is an allo-antibody and, in yet another embodiment, the method can be used for diagnosing alloimmune thrombocytopenia, wherein the detection of the complex is indicative of the presence of alloimmune thrombocytopenia in the subject; for diagnosing fetal and neonatal alloimmune thrombocytopenia (FNAIT), wherein the detection of the complex is indicative of the presence of FNAIT in the subject and/or a gestated offspring of the subject; or for diagnosing post transfusion purpura (PTP), wherein the detection of the complex is indicative of the presence of PTP in the subject. In another embodiment, the antibody is an auto-antibody and, in a further embodiment, the method can be used for diagnosing drug-induced immune thrombocytopenia, wherein the detection of the complex is indicative of the presence of drug-induced immune thrombocytopenia or for diagnosing autoimmune thrombocytopenia, wherein the detection of the complex is indicative of the presence of autoimmune thrombocytopenia in the subject. In such embodiment, the polypeptide can be a soluble polypeptide (such as, for example, ADAMTS13) and in specific embodiments, the method can be used for diagnosing thrombotic thrombocytopenic purpura (TTP), wherein the detection of the complex is indicative of the presence of thrombotic thrombocytopenic purpura in the subject. In some embodiments, the method can comprise further contacting at least one second self-assembled polypeptide immobilized on the surface with the sample, the at least one second self-assembled polypeptide comprising a second ectodomain moiety, and forming a multimer with the at least one first self-assembled polypeptide. In such embodiment, the at least one second self-assembled polypeptide comprises a second chimeric polypeptide non-covalently associated with the first chimeric polypeptide, the second chimeric polypeptide having formula (IIa) or (IIb):

NH₂-SPM-SAAL-SAT-COOH (IIa)

NH₂-SAT-SAAL-SPM-COOH (IIb)

wherein SPM is a second polypeptide moiety derived from the peptide present in the plasma or a fragment thereof; SAAL is an optional second amino acid linker; SAT is a second amino acid tail having at least one acidic amino acid residue each having an R-group comprising a carboxyl group; and — is an amine bond; the carboxyl group of the second chimeric polypeptide is covalently associated to a second silane linker (SSL) moiety, wherein the SSL is covalently associated with at least one second hydroxyl group of the surface; and the FAT is non-covalently associated with the SAT. In some embodiments, the FPM and the SPM are the same, and the at least one first self-assembled polypeptide forms a homomultimer (such as, for example, an homodimer, an homotrimer or a homomultimer comprising additional polypeptide moieties) with the at least one second self-assembled polypeptide. In still another embodiment, the FPM and the SPM are different, and the at least one first self-assembled polypeptide forms a heteromultimer (such as, for example, an heterodimer, an heterotrimer or an heteromultimer comprising additional polypeptide moieties) with the at least one second self-assembled polypeptide. In some embodiments, the surface has the FAAL and/or SAAL. In additional embodiments, the FAT is at least one and up to 50 amino acid residues in length, and has a pI of about 10; and the SAT is at least three and up to 50 amino acid residues in length, and has a pI of about 4. In further embodiments, the FAT has an amino acid sequence of SEQ ID NO: 4 or functional variants or fragments thereof; and the SAT has an amino acid sequence of SEQ ID NO: 9 or functional variants or fragments thereof. In some embodiments, the first chimeric polypeptide comprises a αIIb polypeptide, and the FPM has an amino acid sequence of SEQ ID NO: 2 or functional variants or fragments thereof; the second chimeric polypeptide comprises a β3 polypeptide, and the SPM has an amino acid sequence of SEQ ID NO: 7 or functional variants or fragments thereof. In specific embodiments, one or more of the at least one first self-assembled polypeptide is an activated receptor protein. In another embodiment, the at least one first self-assembled polypeptide comprises a GPlba polypeptide, and the FPM has an amino acid sequence of SEQ ID NO: 11 or functional variants or fragments thereof. In a further embodiment, one or more of the at least one first self-assembled polypeptide is an activated surface protein. In still another embodiment, the at least one first self-assembled polypeptide comprises a αIIb polypeptide, and the FPM has an amino acid sequence of SEQ ID NO: 2 or functional variants or fragments thereof. In a further embodiment, the at least one first self-assembled polypeptide comprises a β3 polypeptide, and the FPM has an amino acid sequence of SEQ ID NO: 7 or functional variants or fragments thereof. In some embodiments, the FSL and/or SSL moiety comprises one or more amine or thiol groups that are covalently associated with the carboxyl groups of the FAT and/or SAT and, in further embodiments, the FSL and/or SSL moiety comprise (3-trimethoxysilylpropyl) diethylenetriamine (DETA). In yet another embodiment, the sample is a blood sample. In some further embodiments, the method comprises detecting the complex by flow cytometry or an enzyme-linked immunosorbent assay.

According to a second aspect, the present disclosure provides a surface for determining the presence of an antibody specific for a polypeptide present in blood or a blood product as described herein. In an embodiment, the surface comprises the at least one first self-assembled polypeptide and the at least one second self-assembled polypeptide, the at least one first self-assembled polypeptide forming a multimer with the at least one second self-assembled polypeptide. In an embodiment, the FPM and the SPM are the same, and the at least one first self-assembled polypeptide forms a homomultimer with the at least one second self-assembled polypeptide. In another embodiment, the FPM and the SPM are different, and the at least one first self-assembled polypeptide forms a heteromultimer with the at least one second self-assembled polypeptide. In another embodiment, the surface comprises a spherical surface. In another embodiment, the surface is a microsphere, such as, for example, a microsphere silica bead. In yet another embodiment, the surface comprises a planar surface.

According to a third aspect, the present disclosure provides a process of immobilizing at least one first self-assembled polypeptide to a surface for diagnosing thrombocytopenia. The surface having at least one first hydroxyl group covalently associated with a first silane linker moiety, the at least one first self-assembled polypeptide comprising a first chimeric polypeptide. The process comprises a) obtaining the first chimeric polypeptide as defined in herien; and b) adding the first chimeric polypeptide to the surface in a solvent under suitable conditions for first chimeric polypeptide to covalently bond to the surface via the first silane linker moiety. In an embodiment, the process further comprises immobilizing at least one second self-assembled polypeptide to the surface, the surfacing further having at least one second hydroxyl group covalently associated with a second silane linker moiety, the at least one second self-assembled polypeptide forming a multimer (such as a dimer or a trimer) with the at least one first self-assembled polypeptide. In such embodiment, the at least one second self-assembled polypeptide comprising a second chimeric polypeptide. In yet another embodiment, the process further comprises obtaining the second chimeric polypeptide as defined herein; and adding the second chimeric polypeptides to the surface in a solvent under suitable conditions for the second chimeric polypeptide to covalently bond to the surface via the second silane linker moieties respectively. In an embodiment, the FPM and the SPM are the same, and the at least one first self-assembled polypeptide forms a homomultimer with the at least one second self-assembled polypeptide. In another embodiment, the FPM and the SPM are different, and the at least one first self-assembled polypeptide forms a heteromultimer with the at least one second self-assembled polypeptide. In some embodiments, the first and/or second silane linker moiety comprises one or more amine or thiol groups that are covalently associated with the carboxyl groups of the FAT and/or SAT. In specific embodiments, the first and/or second silane linker moiety comprises (3-trimethoxysilylpropyl) diethylenetriamine (DETA). In yet another embodiment, the process further comprising coating the surface with the first and/or second silane linker moieties by reacting with the hydroxyl groups. In additional embodiments, the process further comprising obtaining the first chimeric polypeptide and/or the second chimeric polypeptide from recombinant expression in a recombinant host cell. In yet another embodiment, the process further comprising activating the at least one first self-assembled polypeptide and/or at least one second self-assembled polypeptide. In some embodiments, the process further comprises incubating the surface having the first chimeric polypeptide and/or the second chimeric polypeptide bonded thereon in an activation buffer comprising cations. In some embodiments, the activation buffer comprises divalent cations.

According to a fourth aspect, the present disclosure comprises kit for determining the presence of an antibody specific for a peptide present in blood or a blood product. The kit comprising (i) a first chimeric polypeptide as defined herein, wherein the first chimeric polypeptide is capable of binding to an antibody to the first polypeptide moiety and (ii) a surface for covalently associating the first chimeric polypeptide, wherein the surface has first hydroxyl groups covalently associated with a first silane linker moiety. In some embodiments, the kit further comprises a second chimeric polypeptide as defined herein, wherein the first and the second chimeric polypeptide are capable of forming an heteromultimer and wherein the surface further comprises second hydroxyl groups covalently associated with a second silane linker moiety. In another embodiment, the kit further comprises a second chimeric polypeptide as defined herein, wherein the first and the second chimeric polypeptide are capable of forming an homomultimer and wherein the surface further comprises second hydroxyl groups covalently associated with a second silane linker moiety. In an embodiment, the surface comprises a flat surface. In another embodiment, the surface comprises a spherical surface, such as, for example, a microsphere silica bead.

According to a fifth aspect, the present disclosure provides a method of treating thrombocytopenia in a subject. The method comprises detecting the expression of an antibody specific for a polypeptide in blood or a blood product with the method described herein, the surface described herein, or the kit described herein in a sample obtained from a subject suspected of comprising the antibody; and administering a treatment to the subject having been determined to have the antibody specific for the polypeptide in the blood or the blood product. In an embodiment, the method is for treating alloimmune thrombocytopenia, such as fetal an neonatal alloimmune thrombocytopenia (FNAIT). In some embodiments, the treatment comprises administering intravenous immunoglobulin (IVIG), a steroid and/or serial intrauterine platelet transfusions (IUPT). In another embodiment, the method is for treating autoimmune thrombocytopenia, such as, for example, drug induced immune thrombocytopenia, thrombotic thrombocytopenic purpura (TTP) or immune thrombocytopenic purpura (ITP). In an embodiment, the antibody is an anti-ADAMTS13 autoantibody. In another embodiment, the antibody is an anti-GPlba autoantibody. In still a further embodiment, the antibody is an anti-αIIbβ3 autoantibody. In an embodiment, the treatment comprises plasma exchange and/or providing recombinant ADAMTS13. In a further embodiment, the treatment comprises one or more of immunosuppressive agent administration, immunomodulatory agent administration, or splenectomy. In yet another embodiment, the treatment comprises one or more of corticosteroid administration, intravenous immunoglobulin G (IVIG) administration, or anti-RhD therapy.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A to 1C (FIG. 1A) The different conformation states of an integrin are presented. In the inactive (“Bent”) and intermediate (“Extended”) conformations, integrins have no/low affinity towards their target. In the high-affinity ligand binding (“Open”) conformation, integrins have affinity towards their target. For each pair of heterodimer shown, left heterodimer is the α-subunit and the right heterodimer is the β-subunit. (FIG. 1B) Generalized representation of unimolecular self-assembly (SAM) surface assembly. Backbones, each having a tail and a head, assemble with the tails attached to the silica and the heads available for covalent probe attachment. (FIG. 1C) Illustration of the formation of a (3-trimethoxysilylpropyl) diethylenetriamine (DETA) SAM onto a cleaned silica substrate (step 1), preparation of bead immobilization buffer containing recombinant human ectodomain αIIbβ3 having acidic and basic tails (step 2a), and site-specific immobilization of the recombinant human ectodomain αIIbβ3 onto DETA SAMs (step 2b). For each pair of heterodimer shown, left heterodimer is αIIb and the right heterodimer is β3.

FIGS. 2A and 2B show flow cytometry histograms depicting the interaction of bare, DETA coated, inactive αIIbβ3 coupled and activated αIIbβ3 coupled beads with FITC-coupled (FIG. 2A) PSI-E1 (conformation independent αIIbβ3 mAb) and (FIG. 2B) PAC-1 (active conformation dependant αIIbβ3 mAb). The change in mean fluorescent intensity (MFI) is indicated on the X-axis, and the number of events is indicated on the Y-axis in each flow cytometry histogram.

FIG. 3 shows αIIbβ3 is covalently bound to the surface of the beads and covalent binding increases the anti-fouling properties of the αIIbβ3-coupled beads. DETA coated, αIIbβ3 coupled beads with FITC-coupled PSI-E1 (conformation independent αIIbβ3 mAb) in the absence (left panel) and presence (right panel) of SDS. Results are provided as the mean fluorescence intensity on the Y-axis, and adsorbed or covalent αIIbβ3 on the X-axis.

FIGS. 4A to 4C (FIG. 4A) show a schematic depicting the detection of platelet αIIbβ3 autoantibodies using the monoclonal antibody-specific immobilization of platelet antigen (MAIPA) assay. (FIG. 4B) Detection strategy of pathogenic autoantibodies against integrin αIIbβ3 using αIIbβ3 coated beads described herein. (FIG. 4C) Detection of ITP patient autoantibodies from MAIPA confirmed plasma.

FIGS. 5A and 5B compares (FIG. 5A) quantitative flow cytometry fibrinogen assay versus (FIG. 5B) fibrinogen ELISA. n≥3. In FIG. 5A, X-axis is fibrinogen concentration in μM, Y-axis is mean fluorescence intensity. In FIG. 5B, X-axis is fibrinogen concentration in μM, Y-axis is absorbance at 492 nm.

FIG. 6 shows the optimization of the loading of recombinant human αIIbβ3 ectodomain onto the DETA coated silica surface by evaluating the amount of integrin loaded on the surface against the binding activity against its cognate ligand, fibrinogen. Results are shown as flow cytometry signal associate with fibrinogen binding (left axis, grey line and ▪) or with PSI-E1 binding (right axis, black line and) in function of the concentration of αIIbβ3 ectodomain immobilized onto the DETA coated silica surface.

FIG. 7 shows optimization of the loading of different concentrations of recombinant human GPIbα ectodomain onto the surface of 1 μm DETA coated beads, and binding activity detected by NIT F, an antibody against the binding site of human GPIbα.

FIG. 8 shows GPlba coated beads bind to both NIT B and NIT F antibodies that are specific to GPIbα.

FIGS. 9A-D (FIG. 9A) flow cytometry histograms showing binding to negative control anti-CD62p antibodies, (FIG. 9B) corresponding mean fluorescence intensities (MFI) from FIG. 2J plotted in bar graphs, (FIG. 9C) flow cytometry histograms showing binding to negative control anti-GPIBβ antibodies, (FIG. 9D) corresponding mean fluorescence intensities (MFI) from FIG. 2L plotted in bar graphs. In each flow cytometry histogram, the fluorescent intensity is indicated on the X-axis, and the number of events is indicated on the Y-axis. In each bar graph, the particle type is indicated on the X-axis, and the mean fluorescent intensity (MFI) is indicated on the Y-axis.

FIG. 10 shows dose-response curves of flow cytometry assay and MAIPA assay for the detection of anti-αIIbβ3 antibodies (PSI E1) and anti-GPlba antibodies (NIT B).

### DETAILED DESCRIPTION

The present disclosure relates to determining the presence of an antibody in a sample, where the presence of the antibody is associated with a pathological condition, such as thrombocytopenia, in an individual. The antibody is specific to a polypeptide present in the blood or a blood product, such as, for example, plasma. Determining the presence of the antibody involves contacting the sample with one or more of polypeptides immobilized on a surface that maintain their function and active conformation, and detecting the presence or absence of a complex between the antibody and the immobilized polypeptide. The immobilized polypeptides can be in a monomeric form or can form a multimer (homomultimer or heteromultimer). The presence of the complex is indicative of the presence of the antibody in the sample. Ultimately, the presence of the complex is indicative of the presence of the pathological condition in the individual.

The method is designed to detect an antibody which is specific for a polypeptide which is present in blood, plasma or serum. The polypeptide can be a soluble polypeptide present in plasma or serum. The polypeptide can be a cell-associated polypeptide (such as a platelet-associated polypeptide) present in the blood. As used herein “plasma” refers to a cellular-free fraction of blood which has been prevented from clotting. As used herein “serum” refers to a cellular-free fraction of blood which has clotted (and thus no longer includes clotting factors).

The method can be used with a blood sample or a blood-derived sample (such as a serum sample or a plasma sample) suspected of including the antibody. In some embodiments, serum samples are obtained from patients to determine the presence of an antibody in the sample. In some additional embodiments, plasma samples are obtained from patients to determine the presence of an antibody in the sample. In other embodiments, blood samples are obtained from patients to determine the presence of an antibody in the sample.

The antibody are detected by providing and contacting immobilized polypeptides (provided in the form of chimeric polypeptides having an amino acid tail for covalently associating with the surface, thereby immobilizing the chimeric polypeptides to the surface). The immobilized polypeptide can include, in some embodiments, one or more ectodomains (and in some additional embodiments, a complete ectodomain) of a surface/receptor polypeptide. As it is known in the art, an ectodomain is the domain of a membrane protein that extends in the extracellular space. In some embodiments, the ectodomain is involved in binding a ligand and can lead to signal transduction.

In some embodiments, the sample can be an in vitro sample obtained, for example, from culturing cells or tissues. In other embodiments, the sample can be from a subject suspected of having the antibody, and the presence of the antibody is determined for diagnosing a pathological condition or a predisposition to a pathological condition. In some embodiments, determining the presence of an antibody in the sample includes diagnosing the subject for thrombocytopenia, wherein the detection of a complex between the target antibody and an immobilized polypeptide to which the target antibody is specific to, is indicative of the presence of thrombocytopenia in the subject. In some embodiments, the complex is detected by flow cytometry. In other embodiments, the complex is detected by an enzyme-linked immunosorbent assay.

Thrombocytopenia is a condition characterized by abnormally low levels of thrombocytes, or platelets, in the blood. In some embodiments, the antibody is specific to a polypeptide present in blood, plasma or serum. In an embodiment, the antibody is specific for a monomer (or a fragment thereof). In some embodiments, the antibody is specific for a dimer (or a fragment thereof). In some embodiments, the antibody is specific to a homodimer (or a fragment thereof). In some embodiments, the antibody is specific to a heterodimer (or a fragment thereof). When the antibody is specific for a multimeric polypeptide, such as a dimer, it may exhibit specific towards only one of the monomer subunit of the multimer or towards a common epitope formed between the subunits of the multimer.

In some embodiments, the polypeptide present on the surface of a blood cell, such as the platelet, and the antibody is an antibody specific to the polypeptide present on the surface of a platelet. In some embodiments, the antibody is specific to a integrin multimer. In one embodiment, the antibody is specific to a β3 polypeptide of an integrin. In some embodiments, the antibody is specific to a αIIb polypeptide of an integrin multimer. In one embodiment, the antibody is specific for an heterodimer comprising αIIb polypeptide and β3 polypeptide.

In some embodiments, the antibody is specific to a receptor polypeptide present on the surface of a platelet. In some embodiments, the antibody is specific to a glycoprotein present on the surface of a platelet. In one embodiment, the antibody is specific to a GPIbα polypeptide, a αIIb polypeptide or a combination thereof. In such embodiments, determining the presence of the antibody specific to the polypeptide present on the surface of a platelet in the sample includes diagnosing the subject for thrombocytopenia, wherein the detection of the complex is indicative of the presence of thrombocytopenia in the subject.

In some embodiments, the thrombocytopenia is an alloimmune thrombocytopenia and the antibody is an allo-antibody. In such embodiments, determining the presence of an allo-antibody in the sample includes diagnosing the subject for alloimmune thrombocytopenia, wherein the detection of the complex is indicative of the presence of alloimmune thrombocytopenia in the subject.

In one embodiment, the alloimmune thrombocytopenia is a neonatal alloimmune thrombocytopenia. In one embodiment, the alloimmune thrombocytopenia is a fetal and neonatal alloimmune thrombocytopenia (FNAIT), and the antibody is specific to a fetal polypeptide. FNAIT is a condition characterized by an abnormally low platelet count in a fetus' blood, due to the mother's antibodies having been passed via the placenta and attacking the platelets. In one embodiment, a method for diagnosing FNAIT includes determining the presence of an antibody specific to a fetal polypeptide in the maternal blood, plasma or serum or in the newborn's blood, plasma or serum, and the detection of a complex is indicative of the presence of FNAIT in the subject and/or the gestated offspring of the subject (i.e. newborn carried by the subject). In one embodiment, the neonatal polypeptide is a polypeptide present on the surface of a platelet described herein of the fetus, and the antibody is specific to the fetal platelet polypeptide. Antibodies specific for HPA-1a, CD36 and the αIIb integrin have been shown to be present in subjects experiencing FMAIT.

In one embodiment, the thrombocytopenia is post-transfusion purpura (PTP), and the antibody is specific to a transfused allogeneic polypeptide. PTP is a delayed adverse reaction to a blood transfusion or platelet transfusion that occurs when the body has produced alloantibodies to the allogeneic transfused blood or platelets antigens. In one embodiment, a method for diagnosing PTP includes determining the presence of an allo-antibody specific to the allogeneic polypeptide in the plasma, and the detection of a complex is indicative of the presence of PTP in the individual (which can, in some embodiments, be a female subject). In one embodiment, the allogenic polypeptide is a polypeptide present on the surface of a platelet as described herein, and the antibody is specific to the allogenic platelet polypeptide.

In some embodiments, the thrombocytopenia is an autoimmune thrombocytopenia (ITP) and the antibody is an auto-antibody. In some embodiments, the auto-antibody is specific to a polypeptide present on the surface of a platelet. In some embodiments, the auto-antibody is specific to a integrin polypeptide. In one embodiment, the auto-antibody is specific to a β3 polypeptide. In some embodiments, the auto-antibody is specific to an integrin heterodimer. In one embodiment, the auto-antibody is specific to a heterodimer comprising αIIb polypeptide and β3 polypeptide.

In some embodiments, the thrombocytopenia is an autoimmune thrombocytopenia (ITP) and the antibody is an auto-antibody. In some embodiments, the auto-antibody is specific to a glycoprotein present on the surface of a platelet. In one embodiment, the antibody is specific to a GPIbα polypeptide. In such embodiments, determining the presence of an auto-antibody in the sample includes diagnosing the subject for autoimmune thrombocytopenia, wherein the detection of the complex is indicative of the presence of autoimmune thrombocytopenia in the subject.

In some embodiments, the thrombocytopenia is drug-induced immune thrombocytopenia. Drug-induced thrombocytopenia occurs when certain medicines destroy platelets or interfere with the body's ability to make enough platelets. In drug-induced immune thrombocytopenia, the medicine causes the body to produce antibodies which seek and destroy the platelets. In some embodiments, antibodies specific for GPIbα or the integrin αIIbβ3 integrin have been shown to be present in some individuals experiencing drug-induced thrombocytopenia. In one embodiment, the drug-induced immune thrombocytopenia is caused by heparin treatment. Other examples of medicines that causes drug-induced immune thrombocytopenia include: furosemide, gold (for treating arthritis), nonsteroidal anti-inflammatory drugs (NSAIDs), penicillin, quinidine, quinine, ranitidine, sulfonamides, linezolid and other antibiotics, statins.

Specific drugs reported to have a definite causal association with drug-induced immune thrombocytopenia include, but are not limited to, bevacizumab, bortezomib, carfilzomib, cocaine, cyclosporine, docetaxel, everolimus, gemcitabine, imatinib, immune globulin, interferon (alpha, beta, polycarboxylate), ixazomib, mitomycin, muromonab-CD3, oxaliplatin, oxycodone, oxymorphone, palbociblib, penicillin, pentostatin, quetiapine, quinine, sirolimus, sulfisoxazole, sunitinib, tacrolimus, trielina, valproic acid and vincristine.

In some embodiments, the antibody is specific to a soluble polypeptide present in plasma. In some embodiments, the thrombocytopenia is thrombotic thrombocytopenic purpura (TTP), and the antibody is specific to a soluble polypeptide. TTP is a rare blood disorder characterized by clotting in small blood vessels (thromboses), resulting in a low platelet count. In one embodiment, the soluble polypeptide is ADAMTS13. In one embodiment, a method for diagnosing TTP includes determining the presence of an antibody specific to ADAMTS13 in the plasma, and the detection of a complex is indicative of the presence of TTP in the subject.

Immobilized Chimeric Polypeptides

In the context of the present disclosure, polypeptides are provided that are immobilized on surfaces while maintaining their function and, in some embodiments, active conformation. In some embodiments, a surface is provided having hydroxyl groups and at least one self-assembled polypeptide immobilized thereon. The polypeptides are provided in the form of chimeric polypeptides having an amino acid tail for covalently associating with the surface, thereby immobilizing the chimeric polypeptides to the surface.

In some embodiments, the amino acid tail has at least one acid amino acid residue having an R-group comprising a carboxyl group. As used herein, an “acid amino acid residue having an R-group comprising a carboxyl group” refers to natural or unnatural amino acids having a side chain R-group that has one or more terminal or non-terminal carboxyl group (—C(═O)O—), where the carboxyl group is capable of binding with an silane linker moiety. Examples of such natural amino acids ((L)-configuration) having a carboxyl R-group are aspartic acid and glutamic acid. Unnatural amino acids having a side chain R-group include, for example, amino acids with dextrorotary (D)-configuration or amino acids with synthetic or variant R-groups (termed non-natural amino acids) that have been modified to add a terminal or non-terminal carboxyl group. The person skilled in the art will recognized that the amino acid residues present on the tail of each of the chimeric polypeptide is not limited to a particular naturally-occurring or synthetic amino acid residues.

The amino acid tail is attached to the polypeptide (either directly or indirectly using a linker) in such a way that the polypeptide maintains its conformation, functionality or biological activity. In an embodiment, the amino acid tail is attached (either directly or indirectly using a linker) to one end (carboxyl- or amino-end) of the polypeptide. In some embodiments, the amino acid tail is attached to an end of the polypeptide that is opposite to the functional end of the polypeptide to avoid loss of conformation, functionality or biological activity of the polypeptide. For example, if the polypeptide bears its biological activity at the carboxyl-end, the amino acid tail is going to be attached to the amino-end of the polypeptide. In another example, if the polypeptide gears its biological activity at the amino-end, the amino acid tail is going to be attached to the carboxyl-end of the polypeptide. In some embodiments, the amino acid tail is attached to the carboxyl end of the polypeptide. In other embodiments, the amino acid tail is attached to the amino end of the polypeptide.

In order to immobilize the polypeptide on the surface, one of the carboxyl group of the polypeptide (which can be associated with the amino acid tail) is covalently associated with a silane linker. In some embodiments, the carboxyl group of the R-group of one of the amino acid residue of an amino acid tail is for covalent association (a chemical bond) with a silane linker moiety which is immobilized on the surface. In some embodiments, the silane linker moiety has one or more terminal or non-terminal the amine (—NH₂—) or thiol (—S—) groups for covalent association or chemical bonding with the carboxyl group of the polypeptide and, in some embodiments, of the acidic amino acid residue(s) of the amino acid tail associated to the polypeptide.

The polypeptides of the present disclosure can be presented as chimeric polypeptides. In the context of the present disclosure, the chimeric polypeptides can have formula (IIIa) or (IIIb):

NH₂-PM-AAL-AT-COOH (IIIa)

NH₂-AT-AAL-PM-COOH (IIIb)

wherein PM is a polypeptide moiety (which can include an ectodomain), AAL is an optional amino acid linker, and AT is an amino acid tail.

In some embodiments, the chimeric polypeptides can have formula (IVa) or (IVb):

NH₂-PM-AT-COOH (IVa)

NH₂-AT-PM-COOH (IVb)

wherein PM is a polypeptide moiety (which can include an ectodomain) and AT is an amino acid tail.

In some embodiments, the PM includes an ectodomain (and in some additional embodiments, a complete ectodomain) of a surface polypeptide. As it is known in the art, an ectodomain is the domain of a membrane protein that extends in the extracellular space. In some embodiments, the ectodomain is involved in binding a ligand and can lead to signal transduction.

When the amino acid linker (AAL) is absent, the amino acid tail is directly associated with the polypeptide moiety. In the chimeric polypeptide of formula (IVa), this means that the carboxyl terminus of the polypeptide moiety is directly associated (with an amide linkage) to the amino terminus of the amino acid tail. In the chimeric polypeptide of formula (IVb), this means that the carboxyl terminus of the amino acid tail is directly associated (with an amide linkage) to the amino terminus of the polypeptide moiety.

In some embodiments, the presence of an amino acid linker (AAL) is desirable either to provide, for example, some flexibility between the polypeptide moiety and the amino acid tail or to facilitate the construction of the chimeric polypeptide (which can, in some embodiments, be encoded by a nucleic acid molecule). As used in the present disclosure, the “amino acid linker” or “AAL” refer to a stretch of one or more amino acids separating the polypeptide moiety (PM) and the amino acid tail (AT) (e.g., indirectly linking the polypeptide moiety to the amino acid tail). It is preferred that the amino acid linker be neutral, e.g., does not interfere with the biological activity of the polypeptide moiety nor with the biological or chemical activity or interactions of the amino acid tail.

In instances in which the amino acid linker (AAL) is present in the chimeras of formula (IIla or IVa), its amino end is associated (with an amide linkage) to the carboxyl end of the polypeptide moiety and its carboxyl end is associated (with an amide linkage) to the amino end of the amino acid tail. In instances in which the amino acid linker (AAL) is present in the chimeras of formula (IIIb of IVb), its amino end is associated (with an amide linkage) to the carboxyl end of the amino acid tail and its carboxyl end is associated (with an amide linkage) to the amino end of the polypeptide moiety.

Various amino acid linkers exist and include, without limitations, (G)ₙ, (GS)ₙ; (GGS)ₙ; (GGGS)ₙ; (GGGGS)ₙ; (GGSG)ₙ; (GSAT)ₙ, wherein n=is an integer between 1 to 8 (or more). In an embodiment, the amino acid linker is (GGGS)ₙ (also referred to as G₃S) and in still further embodiments, the amino acid linker L comprises more than one G₃S motifs. For example, the amino acid linker can be (G₃S)₃ and have the amino acid sequence of SEQ ID NO: 3.

In some embodiments, the amino acid tail (AT) is at least one amino acid and up to 50 amino acid residues in length. In some embodiments, the amino acid tail (AT) is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues long. In some embodiments, the amino acid tail is no more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues long.

In some embodiments, the at least one self-assembled polypeptide is a receptor protein (which can include one or more ectodomains for the PM). Preferably, the at least one self-assembled polypeptide is an activated receptor protein. In one embodiment, the at least one self-assembled polypeptide is a glycoprotein. In one embodiment, the chimeric polypeptide comprises a GPIbα polypeptide, functional variants or fragments thereof.

In one embodiment, the PM has an amino acid sequence of SEQ ID NO: 11 or variants or fragments thereof.

In some embodiments, the at least one self-assembled polypeptide is a surface protein (which can include one or more ectodomains for the PM). Preferably, the at least one self-assembled polypeptide is an activated surface protein. In some embodiments, at least one self-assembled polypeptide is a platelet surface protein. In one embodiment, the at least one self-assembled polypeptide is an integrin polypeptide. In one embodiment, the first chimeric polypeptide comprises a β3 polypeptide, functional variants or fragments thereof.

In one embodiment, the PM has an amino acid sequence of SEQ ID NO: 7 or variants or fragments thereof.

A variant comprises at least one amino acid difference when compared to the amino acid sequence of the polypeptide polypeptide and exhibits a biological activity substantially similar to the native polypeptide. The polypeptide “variants” have at least 50%, 55%, 60%, 65%, 70%, 75%, 80%, 85%, 90%, 95%, 96%, 97%, 98% or 99% identity to the polypeptide described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The polypeptide “variants” have at least 50%, 55%, 60%, 65%, 70%, 75%, 80%, 85%, 90%, 95%, 96%, 97%, 98% or 99% of the biological activity of the polypeptide described herein. One of the biological activity of the αIIb polypeptide is its ability to non-covalently associated with the β3 polypeptide and bind to its ligand (such as fibrinogen). One of the biological activity of the β3 polypeptide is its ability to non-covalently associated with the αIIb polypeptide and bind to its ligand (such as the fibrinogen). The biological of “variants” of the αIIb or the β3 can be assessed by various means known in the art, including, but not limited to antibody-based techniques (flow cytometry, ELISA assay for example) as well as microscopy techniques.

The variant polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological functions of the enzyme. A substitution, insertion or deletion is said to adversely affect the polypeptide when the altered sequence prevents or disrupts a biological function associated with the enzyme. For example, the overall charge, structure or hydrophobic-hydrophilic properties of the polypeptide can be altered without adversely affecting a biological activity. Accordingly, the amino acid sequence can be altered, for example to render the peptide more hydrophobic or hydrophilic, without adversely affecting the biological activities of the enzyme.

The polypeptide can be a fragment of polypeptide or fragment of a variant polypeptide. A polypeptide fragment comprises at least one less amino acid residue when compared to the amino acid sequence of the possesses and still possess a biological activity substantially similar to the native full-length polypeptide or functional variants thereof Polypeptide “fragments” have at least at least 100, 200, 300, 400, 500, 600, 700, 800, 900 or more consecutive amino acids of the polypeptide or the polypeptide variant. The polypeptide “fragments” have at least 50%, 55%, 60%, 65%, 70%, 75%, 80%, 85%, 90%, 95%, 96%, 97%, 98% or 99% identity to the polypeptide described herein. The polypeptide “fragments” have at least 50%, 55%, 60%, 65%, 70%, 75%, 80%, 85%, 90%, 95%, 96%, 97%, 98% or 99% of the biological activity of the polypeptides and the functional fragments described herein. In some embodiments, fragments of the polypeptides can be employed for producing the corresponding full-length enzyme by peptide synthesis. Therefore, the fragments can be employed as intermediates for producing the full-length polypeptides.

Immobilized Chimeric Multimer Polypeptides

In some embodiments, at least one first self-assembled polypeptide are immobilized on a surface. The at least one first self-assembled polypeptide comprises a first chimeric polypeptide is of formula (Ia) or (Ib):

NH₂-FPM-FAAL-FAT-COOH (Ia)

NH₂-FAT-FAAL-FPM-COOH (Ib)

wherein FPM is a first polypeptide moiety; FAAL is an optional first amino acid linker; FAT is a first amino acid tail having at least one acidic amino acid residues having an R-group comprising a carboxyl group; and — is an amine bond.

In some embodiments, the first chimeric polypeptide is of formula (Va) or (Vb):

NH₂-FPM-AAT-COOH (Va)

NH₂-AAT-FPM-COOH (Vb)

wherein FPM is a first polypeptide moiety; FAT is a first amino acid tail having at least one acidic amino acid residues having an R-group comprising a carboxyl group; and the — is an amine bond.

In some embodiments, the FPM includes an ectodomain (and in some additional embodiments, a complete ectodomain) of a surface polypeptide. As it is known in the art, an ectodomain is the domain of a membrane protein that extends in the extracellular space. In some embodiments, the ectodomain is involved in binding a ligand and can lead to signal transduction.

In instances in which the first amino acid linker (FAAL) is present in the chimeras of formula (Ia), its amino end is associated (with an amide linkage) to the carboxyl end of the first polypeptide moiety and its carboxyl end is associated (with an amide linkage) to the amino end of the first amino acid tail. In instances in which the first amino acid linker (FAAL) is present in the chimeras of formula (IIb), its amino end is associated (with an amide linkage) to the carboxyl end of the first amino acid tail and its carboxyl end is associated (with an amide linkage) to the amino end of the first polypeptide moiety.

When the first amino acid linker (FAAL) is absent, the first amino acid tail is directly associated with the first polypeptide moiety. In the chimeric polypeptide of formula (Va), this means that the carboxyl terminus of the first polypeptide moiety is directly associated (with an amide linkage) to the amino terminus of the first amino acid tail. In the chimeric polypeptide of formula (Vb), this means that the carboxyl terminus of the first amino acid tail is directly associated (with an amide linkage) to the amino terminus of the first polypeptide moiety.

In some embodiments, at least one second self-assembled polypeptide are immobilized on a surface. The at least one second self-assembled polypeptide comprises a second chimeric polypeptide of formula (IIa) or (IIb):

NH₂-SPM-SAAL-SAT-COOH (IIa)

NH₂-SAT-SAAL-SPM-COOH (IIa)

wherein SPM is a second polypeptide moiety; SAAL is an optional second amino acid linker; SAT is a second amino acid tail having at least one acid amino acid residue having an R-group comprising a carboxyl group; the — is an amine bond.

In some embodiments, the second chimeric polypeptide is of formula (VIa) or (VIb):

NH₂-SPM-SAT-COOH (VIa)

NH₂-SAT-SPM-COOH (VIa)

wherein SPM is a second polypeptide moiety; SAT is a second amino acid tail having at least one acid amino acid residue having an R-group comprising a carboxyl group; and the — is an amine bond.

In some embodiments, the SPM includes an ectodomain (and in some additional embodiments, a complete ectodomain) of a surface polypeptide. As it is known in the art, an ectodomain is the domain of a membrane protein that extends in the extracellular space. In some embodiments, the ectodomain is involved in binding a ligand and can lead to signal transduction.

In instances in which the second amino acid linker (SAAL) is present in the chimeras of formula (IIa), its amino end is associated (with an amide linkage) to the carboxyl end of the second polypeptide moiety and its carboxyl end is associated (with an amide linkage) to the amino end of the second amino acid tail. In instances in which the second amino acid linker (SAAL) is present in the chimeras of formula (IIb), its amino end is associated (with an amide linkage) to the carboxyl end of the second amino acid tail and its carboxyl end is associated (with an amide linkage) to the amino end of the second polypeptide moiety.

When the second amino acid linker (SAAL) is absent, the second amino acid tail is directly associated with the second polypeptide moiety. In the chimeric polypeptide of formula (VIa), this means that the carboxyl terminus of the second polypeptide moiety is directly associated (with an amide linkage) to the amino terminus of the second amino acid tail. In the chimeric polypeptide of formula (VIb), this means that the carboxyl terminus of the second amino acid tail is directly associated (with an amide linkage) to the amino terminus of the second polypeptide moiety.

In some embodiments, at least one first self-assembled polypeptide and at least one second self-assembled polypeptide are immobilized on a surface, wherein the at least one first self-assembled polypeptide and the at least one second self-assembled polypeptide forms a dimer (such as a homodimer or an heterodimer). The FAT is non-covalently associated with the SAT.

In some embodiments, the FPM and the SPM are the same, and the at least one first self-assembled polypeptide and the at least one second self-assembled polypeptide forms a homomultimer such as a homodimer or a homotrimer. In some embodiments, the FPM and the SPM are different, and the at least one first self-assembled polypeptide and the at least one second self-assembled polypeptide forms a heteromultimer such as a heterodimer or a heterotrimer.

In some embodiments, the FAT is an acidic amino acid tail. In one embodiment, the FAT has a pI between about 3 and 5.

In some embodiments, the SAT is a basic amino acid tail. In one embodiment, The BAT has a pI between about 9 and 11

As used herein, an “acidic amino acid tail” refers to an amino acid tail having one or more acidic amino acid residues, such that the pl of the acidic amino acid tail is less than 7. Examples of acidic amino acid residues include: aspartic acid and glutamic acid. In some embodiments, the pI of the acidic amino acid tail is less between about 3 and 5. In one embodiment, the pI of the acidic amino acid tail is about 4, more preferably 3.91. The acidic amino acid tail has a charge which will allow it to interact non-covalently with the basic amino acid tail (described below) and place the FPM is an active conformation.

In some embodiments, the acidic amino acid tail (AAT) is at least three amino acid and up to 50 amino acid residues in length. In some embodiments, the acidic amino acid tail (AAT) is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues long. In some embodiments, the acidic amino acid tail is no more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4 or 3 amino acid residues long. In some embodiments, the AAT has an amino acid sequence of SEQ ID NO: 4 or variants or fragments thereof.

As used herein, a “basic amino acid tail” refers to an amino acid tail having one or more basic amino acid residues, such that the pl of the basic amino acid tail is greater than 7. Examples of basic amino acid residues include: arginine, histidine, and lysine. In some embodiments, the pI of the basic amino acid tail is less between about 9 and 11. In one embodiment, the pI of the acidic amino acid tail is about 10, more preferably 10.05. The basic amino acid tail has a charge which will allow it to interact non-covalently with the acidic amino acid tail and place the SPM is an active conformation.

In some embodiments, the basic acid tail (BAT) is at least one amino acid and up to 50 amino acid residues in length. In some embodiments, the basic amino acid tail (BAT) is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues long. In some embodiments, the basic amino acid tail is no more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues long. In some embodiments, the BAT has an amino acid sequence of SEQ ID NO: 9 or variants or fragments thereof.

To immobilize chimeric polypeptides to the surface, one or more of the carboxyl group of the acidic amino acid tail (AAT) of the first chimeric polypeptide is covalently associated to a first silane linker (FSL) moiety, which in turn is covalently associated with a first hydroxyl group of the surface. One or more of the carboxyl group of the basic amino acid tail (BAT) of the second chimeric polypeptide is covalently associated to a second silane linker (SSL) moiety, which in turn is covalently associated with a second hydroxyl group of the surface. When immobilized on the surface, the acidic amino acid tail (AAT) is non-covalently associated (such as charge-charge interaction) with the basic amino acid tail (BAT). Furthermore, the first polypeptide moiety is non-covalently associated with the second polypeptide moiety. In some embodiments, the first polypeptide moiety is non-covalently associated with the second polypeptide moiety in an active conformation.

In some embodiments, the first chimeric polypeptide has the FAAL, and/or the second chimeric polypeptide has the SAAL. In some embodiments, the first chimeric polypeptide has the FAAL, and the second chimeric polypeptide has the SAAL. In some embodiments, the first chimeric polypeptide has the FAAL, but the second chimeric polypeptide does not have the SAAL. In some embodiments, the first chimeric polypeptide does not have the FAAL, but the second chimeric polypeptide has the SAAL.

In some embodiments, the first chimeric polypeptide is of formula (Ia) and the second chimeric polypeptide is of formula (IIa). In some embodiments, the first chimeric polypeptide is of formula (Ib) and the second chimeric polypeptide is of formula (IIa). In some embodiments, the first chimeric polypeptide is of formula (Ia) and the second chimeric polypeptide is of formula (IIb). In some embodiments, the first chimeric polypeptide is of formula (Ib) and the second chimeric polypeptide is of formula (IIb).

In some embodiments, the first chimeric polypeptide is of formula (Va) and the second chimeric polypeptide is of formula (VIa). In some embodiments, the first chimeric polypeptide is of formula (Vb) and the second chimeric polypeptide is of formula (VIa). In some embodiments, the first chimeric polypeptide is of formula (Va) and the second chimeric polypeptide is of formula (VIb). In some embodiments, the first chimeric polypeptide is of formula (Vb) and the second chimeric polypeptide is of formula (VIb).

In some embodiments, the at least one self-assembled first and second polypeptides are a surface protein (which can include one or more ectodomains for the FPM and/or the SPM). Preferably, the at least one self-assembled first and second polypeptides are activated surface proteins. In some embodiments, at least one self-assembled first and second polypeptides are platelet surface proteins. In one embodiment, the at least one self-assembled first and second polypeptides are an integrin dimer. In one embodiment, the first chimeric polypeptide comprises a αIIb polypeptide, functional variants or fragments thereof; and the second chimeric polypeptide comprises a β3 polypeptide, functional variants or fragments thereof.

In one embodiment, the FPM has an amino acid sequence of SEQ ID NO: 2 or variants or fragments thereof. In such embodiment, the corresponding first chimeric polypeptide can have, for example, the amino acid sequence of SEQ ID NO: 1 or variants thereof or fragments thereof. In another embodiment, the FPM has an amino acid sequence of SEQ ID NO; 7 or variants thereof or fragments thereof.

In one embodiment, the SPM has an amino acid sequence of SEQ ID NO: 2 or variants or fragments thereof. In another embodiment, the SPM has an amino acid sequence of SEQ ID NO: 7 or variants thereof or fragments thereof. In such embodiment, the corresponding second chimeric polypeptide can have, for example, the amino acid sequence of SEQ ID NO: 6 or variants thereof or fragments thereof.

Organosilane Surfaces

In the context of the present disclosure, the carboxyl groups of the chimeric polypeptides are covalently associated to silane linker moieties, which in turn are covalently associated with hydroxyl groups of the surface. In some embodiments, the surface is made of a material such as silica, glass, metal, or plastics. In some embodiments, the surface has terminal hydroxyl groups. In other embodiments, the surface is chemically treated to add terminal hydroxyl groups. The grafting density of hydroxyl groups on the surface can be adjusted, as known in the art, so as to favor or allow the non-covalent association of the acidic and basic amino acid tails as well as the non-covalent association of the first polypeptide moiety and the second polypeptide moiety.

In some embodiments, the surface is curved. In one embodiment, the surface is spherical. In one embodiment, the surface is a microsphere. In one embodiment, the microsphere is a silica bead. In one embodiment, the microsphere is a glass bead. In one embodiment, the microsphere is a metal bead. In one embodiment, the microsphere is a plastic bead.

In some embodiments, the surface has a planar surface. In some embodiment, the surface is flat. In one embodiment, the surface is a film. In other embodiments, the surface is a platform. In additional embodiments, the surface is a flat silica surface, a flat glass surface, a flat plastic surface or a flat metal surface.

In some embodiments, the hydroxyl groups of the surface is covalently associated with the silicone atom of a silane linker moiety. A silane is an inorganic compound having the chemical formula, SiH4. As used herein a “silane linker” refers to a compound based on SiH4, where one or more of the hydrogens is substituted with a group having one or more terminal and/or non-terminal the amine (—NH2-) or thiol (—S—) groups. The terminal and/or non-terminal the amine (—NH2-) or thiol (—S—) groups of a silane linker moiety is covalently associated with the carboxyl groups of the acidic or basic tails.

In some embodiments, the silane linker moiety is an amino silane. In one embodiment, the amino silane is an amino alkyl silane. In one embodiment, the silane linker moiety is 3-trimethoxysilylpropyl) diethylenetriamine (DETA). In some embodiments, the silane linker moiety is an thiol silane. In one embodiment, the amino silane is a thiol alkyl silane.

In embodiments where the first chimeric polypeptide comprises a αIIb polypeptide, variants or fragments thereof; and the second chimeric polypeptide comprises a β3 polypeptide, variants or fragments thereof, the surface is a probe surface such as a αIIbβ3 coupled bead, film, or platform. The αIIbβ3 coupled bead, film, or platform has application as a molecular probe to identify integrin binding partners, and active conformation of the αIIbβ3 heterodimer is maintained to allow for binding with platelets to form platelet aggregates.

Processes for Immobilizing Chimeric Polypeptides

In the context of the present disclosure processes of immobilizing at least one self-assembled polypeptide to a surface is provided. In some embodiments, processes of immobilizing at least one self-assembled first and second polypeptides to a surface is provided. The at least one self-assembled first and second polypeptides forms a multimer such as a dimer or a trimer. In some embodiments, the first polypeptide moiety and the second polypeptide moiety form a multimer in an active conformation. In some embodiments, the multimer is a homodimer or a homotrimer. In other embodiments, the multimer is a heterodimer or a heterotrimer.

In some embodiments, the surface has hydroxyl groups covalently associated with a silane linker moiety, and the at least one self-assembled polypeptide comprises a chimeric polypeptide. In some embodiments, the process includes obtaining a chimeric polypeptide as described herein, and adding the first chimeric polypeptide to the surface in a solvent under suitable conditions for the first chimeric polypeptide to covalently bond to the surface via a silane linker moiety.

In some embodiments, the surface has hydroxyl groups covalently associated with a first and a second silane linker moiety, and the at least one self-assembled first and second polypeptides comprise a first and a second chimeric polypeptide, respectively. In some embodiments, the process includes obtaining a first chimeric polypeptide as described herein, obtaining a second chimeric polypeptide as described herein, and adding the first and second chimeric polypeptide to the surface in a solvent under suitable conditions for the first and second chimeric polypeptides to covalently bond to the surface via a silane linker moiety, wherein the first polypeptide moiety is non-covalently associated with the second polypeptide moiety. In some embodiments, the first polypeptide moiety and the second polypeptide moiety form a multimer (such as a dimer or a trimer) in an active conformation.

In some embodiments, the process involves coating the surface with a silane linker by reacting with the hydroxyl groups of the surface. In some embodiments, the surface having hydroxyl groups are coated with a silane linker having one or more terminal and/or non-terminal amine (—NH2-) or thiol (—S—) groups. In one embodiment, the silane linker is DETA and the process involves coating the DETA.

As it is known in the art, the process can include pre-treating the surface to provide hydroxyl groups to allow the silane linker to associate with the surface. This can be done, for example, by pre-treating the surface with piranha (70%H2SO4+30% of 30% H2O2), 20-40% NaOH/KOH, or another strong acid or base treatment. The person skilled in the art will recognize that any pre-treatment exposing hydroxyl groups on the surface can be used in the context of the present disclosure.

In some embodiments, the process involves the recombinant expression of the first and/or the second chimeric polypeptide in a recombinant host cell to obtain the first and/or the second chimeric polypeptide. In such embodiment, the process can also include a step of purifying, at least partially, the first and/or second chimeric polypeptide from the recombinant host. In some embodiments, the process involves recombinant expression of a surface protein, which can include its ectodomain. In one embodiment, the process involves the recombinant expression of an integrin dimer.

In some embodiments, the process involves activating the multimer (such as the dimer or the trimer) form between the first and second chimeric polypeptide. For example, the process can include incubating the surface having the first and second chimeric polypeptides bonded thereon in an activation buffer (which can, in some embodiments, comprise cations). In some embodiments, the cations are divalent cations. In one embodiment, the cations are magnesium cations (Mg2+). In one embodiment, the cations are calcium cations (Ca2+). In one embodiment, the cations are manganese cations (Mn2+).

Kits and Treatments

In the context of the present disclosure, kits for determining the presence of an antibody specific for a peptide present in plasma are provided. The kit has a first chimeric polypeptide as described herein for binding to the antibody, and a surface for immobilizing the first chimeric polypeptide as described herein.

In some embodiments, the kit further comprises a second chimeric polypeptide which forms a multimer with the first chimeric polypeptide, and the surface is for further immobilizing the second chimeric polypeptide as described herein. In some embodiments, the first polypeptide moiety and the second polypeptide moiety form a multimer in an active conformation. In some embodiments, the multimer is a homodimer or a homotrimer. In other embodiments, the mutlimer is a heterodimer or a heterotrimer.

In the context of the present disclosure, methods of treating thrombocytopenia in a patient using the methods, processes, and kits described herein are provided. In some embodiments, treating thrombocytopenia in a patient includes detecting the expression of an antibody specific for a polypeptide in the blood, plasma or serum as described herein in a sample obtained from a subject suspected of comprising the antibody, and administering a treatment to the subject having been determined to have the antibody specific for the peptide in the plasma.

In some embodiments, methods for treating alloimmune thrombocytopenia are provided. In one embodiment, methods for treating fetal and neonatal alloimmune thrombocytopenia (FNAIT) are provided, comprising one or more of: maternal intravenous immunoglobulin (IVIG), maternal steroid administration, or serial intrauterine platelet transfusions (IUPT).

In some embodiments, methods for treating autoimmune thrombocytopenia are provided. In one embodiment, methods for treating drug induced immune thrombocytopenia are provided. In one embodiment, methods for treating immune thrombocytopenic purpura (ITP) are provided. The treatment includes one or more of immunosuppressive agent administration, immunomodulatory agent administration, or splenectomy. For example, treatment includes one or more of corticosteroid administration, intravenous immunoglobulin G (IVIG) administration, or anti-RhD therapy.

In one embodiments, methods for treating thrombotic thrombocytopenic purpura (TTP) are provided, comprising plasma exchange and/or providing recombinant ADAMTS13.

The present invention will be more readily understood by referring to the following examples which are given to illustrate the invention rather than to limit its scope.

### EXAMPLE I

### Immobilization of αIIbβ3 on Silica Beads

Materials. Unless otherwise specified, all reagents were purchased from Sigma-Aldrich™ and used as received. Furthermore, all buffers and aqueous solutions were prepared using ultrapure distilled deionized water (ddH₂O) with a measured resistivity ≥18.0 MΩ·cm.

DETA Silanization of Ferromagnetic Silica Beads and Subsequent Immobilization of αIIbβ3. Unless otherwise specified rinsing of ferromagnetic silica beads consists of magnetically pelleting the beads, removing the supernatant and resuspending in new solution. (—OH). Activated ferromagnetic silica beads were purchased from Magna Medics™ and used as received. The beads were first rinsed (3×) in spectral grade methanol, sonicated for 5 min then rinsed one last time with spectral grade methanol. The rinsing procedure was then repeated with toluene. Following the rinsing procedure, the beads were dried for 2 h at 180° C. then placed in an 80% humidity chamber overnight. In a glovebox under N₂ atmosphere a 1% (v/v) solution of neat DETA diluted in anhydrous toluene was prepared in an OTS silanized glass vial. In an OTS silanized 20 mL scintillation vial silica beads were then immersed in this solution, to a final volume equal to that which was originally aliquoted from the activated bead stock solution, capped and incubated at room temperature on a bench top oscillator overnight. The freshly silanized beads were then rinsed (3×) with anhydrous toluene, sonicated for 5 min and rinsed again. The rinsing procedure was repeated with spectral grade methanol and finally PBS. Beads were then taken up in PBS at a concentration of approximately 1×10⁸ beads/mL.

Recombinant human ectodomain αIIbβ3 or GPIbα was freshly immobilized onto silanized beads as experimentally required. Bead immobilization buffer was prepared (4 mM EDC, 10 mM sulfo-NHS, 10 mM sodium phosphate, 140 mM NaCl, 5 mM KCl pH=7.4) and recombinant human extracellular αIIbβ3 was added to a final concentration of 125 μg/mL. Immediately after the preparation immobilization buffer, freshly silanized beads were taken up in immobilization buffer to a final bead concentration of approximately 1×10⁸ beads/mL, and incubated at 4° C. overnight on a bench top oscillator. The reaction was quenched by adjusting the pH to 8.5 and incubating at RT for 1 h. The beads were then rinsed (3×) with copious amounts of PBS, sonicated for 5 minutes then rinsed one last time with PBS, the concentration was then adjusted to approximately 1×10⁸ beads/mL and then stored at 4° C. in a screw top vial until needed.

Activation of αIIbβ3 SAMs. Activation of SAM immobilized integrin αIIbβ3 was achieved by 72 h incubation in activation buffer (1 mM each of CaCl₂, MnCl₂ and MgCl₂ taken up in PBS).

X-ray Photoelectron Spectroscopy. Angle-resolved X-ray photoelectron spectroscopy (XPS) to evaluate substrate silanization (SAM formation) and subsequent αIIbβ3 immobilization was performed with a Theta probe XPS Instrument (ThermoFisher Scientific) located at Surface Interface Ontario (University of Toronto, Toronto, Ontario, Canada). Quartz surfaces were analyzed with monochromated Al Kα X-rays at takeoff angles of 27.5, 42.5, 57.5, and 72.5° relative to the normal. The binding energy scale was calibrated to the C1s signal at 285 eV. Peak fitting and data analysis were performed with the Avantage Data System software package (ThermoFisher Scientific™) provided with the instrument. Complete XPS data are tabulated Table 1.

TABLE 1
X-ray photoelectron spectroscopy (XPS) analysis of bare, DETA
silanized and αllbβ3 immobilized magnetic silica beads. (n ≥ 3)

| | Carbon | Nitrogen | Oxygen | Silicon |
|---|---|---|---|---|
| Surface | 293-280 eV | 405-395 eV | 543-527 eV | 103-98 eV |
| Bare | 11.08 ± 1.83^{a} | 4.79 ± 0.77 ^{a} | 57.49 ± 7.09 | 26.63 ± 3.02 |
| DETA | 37.02 ± 2.94 | 12.60 ± 3.29 | 35.91 ± 2.91 | 14.47 ± 1.82 |
| αllbβ3 | 40.79 ± 2.65 | 11.04 ± 2.81 | 38.03 ± 6.26 | 10.14 ± 0.94 |

^{a} Unavoidable adventitious carbon and nitrogen contamination

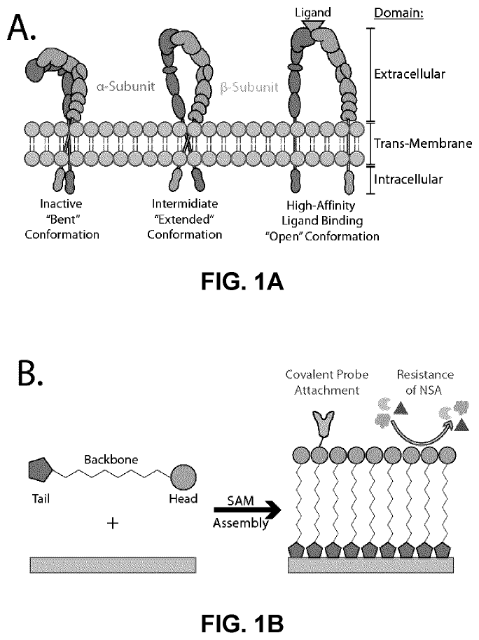

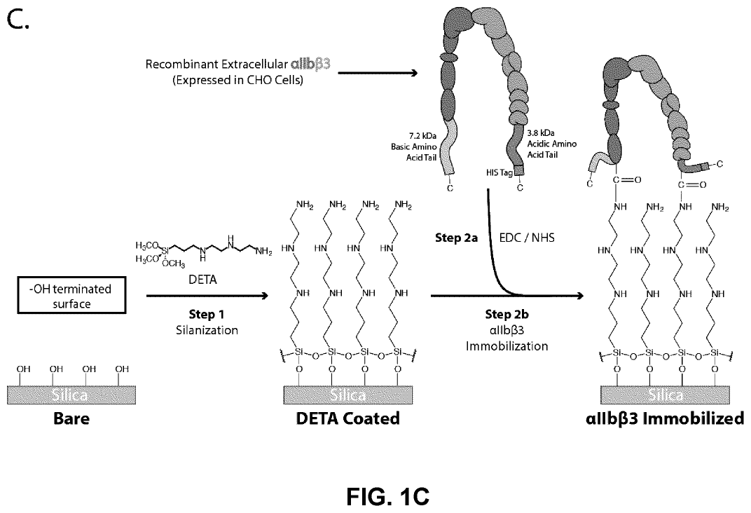

Preparation of αIIbβ3 Coated Beads. Ferromagnetic silica beads were prepared upon formation of DETA adlayers on cleaned silica beads followed by covalent immobilization of αIIbβ3 (FIG. 1C). Each step of surface preparation was characterized using X-ray photoelectron spectroscopy (XPS) by following the evolution of the characteristic elements of silica (Si and O), DETA and αIIbβ3 (C and N), (see Table 1). Beside the small signal attributed to unavoidable adventitious carbon and nitrogen contamination, bare silica showed signals for oxygen and silicon at an approximate 2:1 ratio, as would be expected for quartz (SiO2). Following DETA silanization, signals appeared for carbon and nitrogen, in contrast, the oxygen and silicon signals (that mainly originate from the now underlying silica substrate) decreased. αIIbβ integrin was then covalently coupled to the DETA surface via carbodiimide EDC/NHS chemistry. Following this step XPS signals for carbon, nitrogen and oxygen remained essentially unchanged while the silicon signal was significantly attenuated, indicating further burying of the silica substrate by αIIbβ3.

Co-aggregation of αIIbβ3 Coated Beads. Briefly, 1×10⁸ platelets/mL were incubated with 1×10⁸ αIIbβ3-coupled beads /mL and platelet aggregation was initiated with 5 U/mL of thrombin. After allowing aggregation to proceed for 45 minutes, the beads were magnetically pulled down and washed. The αIIbβ3-coupled beads pulled down whole platelet aggregates while the DETA beads did not (data not shown), indicating that the surface immobilized integrin is present and biologically active.

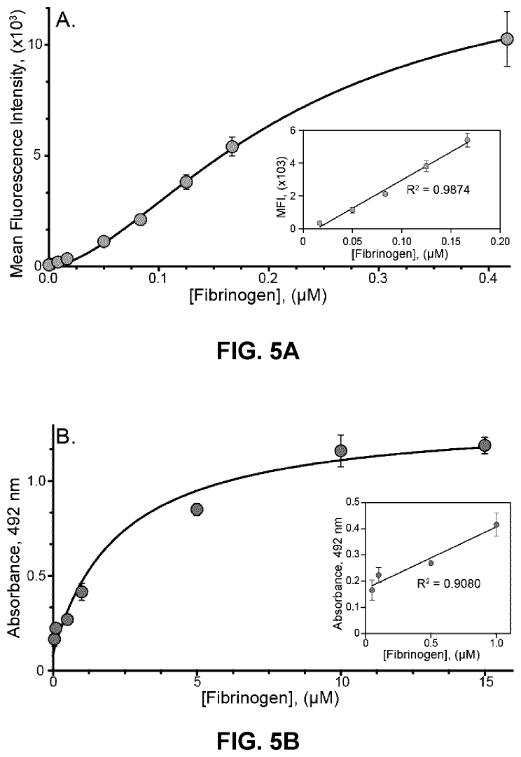

Loading optimization. At various immobilization concentrations of αIIbβ3 integrin, the resulting αIIbβ3-coupled beads were evaluated for fibrinogen or PSI-E1 binding using flow cytometry. PSI-E1 is an antibody that binds linear epitope and indicated represents the amount of integrin loaded onto the surface. While, fibrinogen is the natural ligand of αIIbβ3, and fibrinogen binding indicates the activity of the αIIbβ3-coupled beads. The optimal loading concentration was determined to be 250 μg/mL, increasing the concentration, although increases the amount of αIIbβ3 integrin on the bead surface does not increase the binding activity of the resulting αIIbβ3-coupled beads (FIGS. 5A and 5B).

Flow-Cytometric Analysis of Ferromagnetic Silica Beads. Unless otherwise stated, all flow cytometry experiments were conducted under the same conditions using either a BD FACS Calibur™ or BD Fortessa™ X20 flow cytommeter. 3 μL of 1×10⁸ beads/mL were mixed with the desired concentration of fluorescently labelled antibody or ligand to a final volume of 100 μL in PBS and incubated for 1 h. Following incubation, the samples were diluted to a final volume of 1 mL and flow cytometrically analyzed. Samples that were compared with one another were run on the same instrument under the same instrumental conditions (signal gain, flow rate etc.). Any differences in absolute mean fluorescence intensity (MFI) value between different experiments was due to variance between the two.

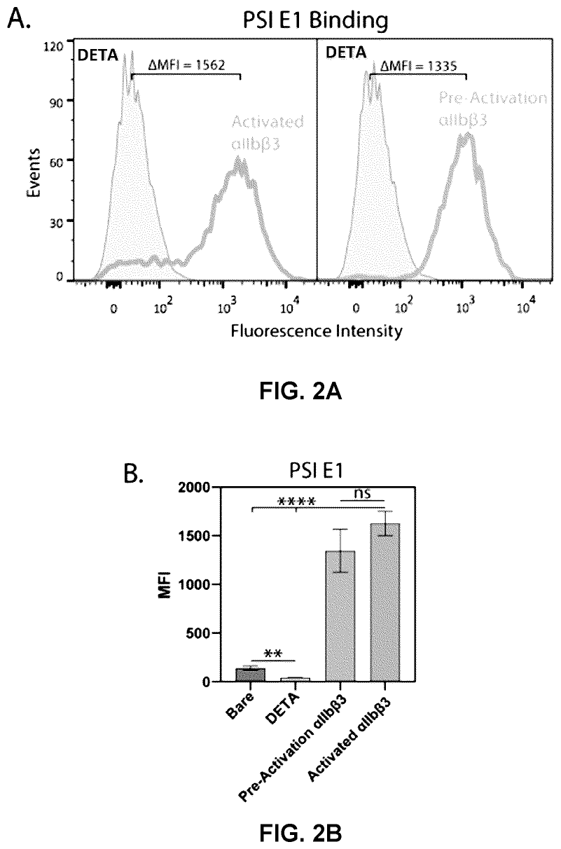

αIIbβ3 Coupled Bead Flow Cytometry Analysis: Although Flow cytometry was utilized to analyze the beads for the presence of the integrin. αIIbβ3 coupled and DETA coated beads were incubated with FITC coupled PSI-E1, an antibody against linear epitope of the PSI-domain of β3 integrin. Upon analysis with flow cytometry, (FIG. 2A) and indeed the integrin is present on the bead surface. Integrin function (ligand binding) is highly conformation dependent (FIG. 1A), and the intrgrin must be induced into the high affinity upright conformation prior to ligand binding. αIIbβ3 was activated by incubation with a mixture of divalent cations (Mg²⁺, Ca²⁺ and Mn²⁺) in PBS buffer. After the activation step, the beads were analysed with flow cytometry for binding of Alexa488™ labelled fibrinogen (native αIIbβ3 ligand) and PAC-1 (an antibody specific for the active upright conformation of αIIbβ3) (See Table 2, FIG. 2B). The data revealed, FIGS. 2A and 2B, that indeed the covalently coupled integrin adopted the high-affinity ligand binding conformation, furthermore, the DETA coating was able to markedly resist the non-specific binding of sticky fibrinogen, a protein known to be highly fouling and clog up blood oxygenators and dialysis equipment REF, without a blocking step (i.e. BSA) when compared to the uncoated silica beads.

TABLE 2
Flow cytometry determined mean fluorescent intensities (MFI)
of bare, DETA and activated/inactive aIIbβ3 beads bound (or nor)
with alexa488-fibrinogen (endogenerous ligand), FITC-PSI E1
(confirmation independent anti-aIIbβ3 antibody), FITC-antiCD62
and FITC-anti GPIbβ (Isotype controls of the various antibodies).
n ≥ 3

| | |
|---|---|
| | Mean Fluorescence |
| Beads | Intensity (MFI) |

50 nM Alexa 488 - Fibrinogen

| | |
|---|---|
| Bare | 577 ± 18 |
| DETA | 74.6 ± 5.2 |
| Inactive allbβ3 | 52.7 ± 3.9 |
| Activated allbβ3 | 2383 ± 18 |

50 nM FITC - PAC 1

| | |
|---|---|
| Inactive allbβ3 | 212 ± 29 |
| Activated allbβ3 | 942 ± 28 |

50 nM FITC - PSI E1

| | |
|---|---|
| Bare | 139 ± 12 |
| DETA | 41.7 ± 3.1 |
| Inactive allbβ3 | 1343 ± 21 |
| Activated allbβ3 | 1625 ± 19 |

100 nM FITC-anti CD62p

| | |
|---|---|
| DETA | 52.7 ± 3.7 |
| Inactive allbβ3 | 54.9 ± 6.0 |
| Activated allbβ3 | 56.5 ± 4.9 |

100 nM FITC-anti GPIbβ

| | |
|---|---|
| DETA | 36.0 ± 5.1 |
| Inactive allbβ3 | 36.7 ± 6.9 |
| Activated allbβ3 | 40.6 ± 6.5 |

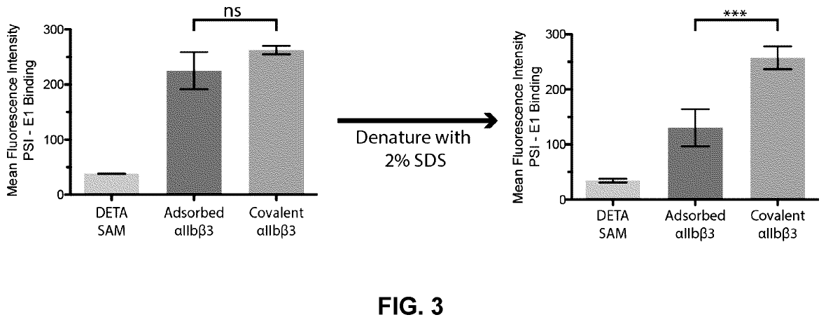

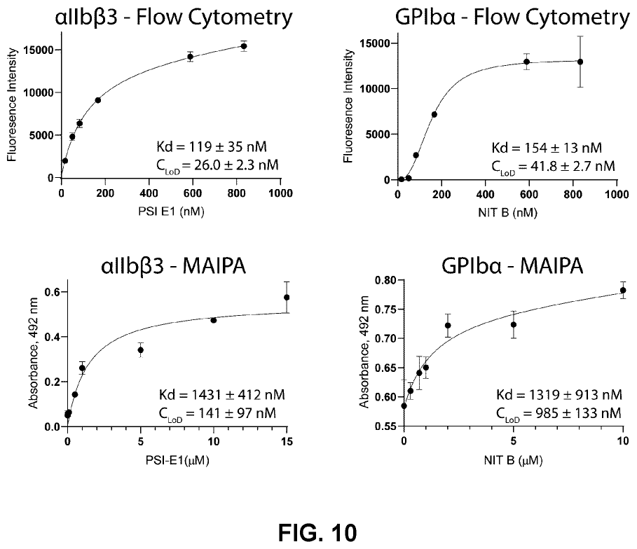

However, this data does not indicate if αIIbβ3 is covalently bound to the DETA coated bead. To evaluate this, beads were prepared in the same manner only lacking the carbodiimide crosslinker (EDC/NHS, FIG. 10 step 2) and αIIbβ3 was allowed to electrostatically and non-spedifically adsorb onto the bead surface. PSI-E1 flow cytometry analysis (FIG. 3) revealed that initially both the covalently bound and adsorbed beads loaded approximately the same amount of αIIbβ3. However, the addition of 2% SDS reduced the adsorbed bead signal by approximately 50% while the covalent bead signal remained the same, indicating that αIIbβ3 was indeed covalently bound to DETA. The adsorbed and covalently bound αIIbβ3 beads were activated and analyzed for fibrinogen (Fg) binding, and despite similar loading of αIIbβ3, the covalently bound bead produced a markedly higher signal (FIG. 3). These data indicated that covalent attachment of αIIbβ3 enhances the active binding conformation of the integrin.

### EXAMPLE II

### Detection of Immune Thrombocytopenia (ITP) Autoantibodies

The current gold standard assay for the detection of anti-platelet auto- and allo-antibodies is the MAIPA assay. The assay is complex, time consuming, lacks standardization and is prone to false positives and negatives, depending on the reagents used. Briefly, intact washed platelets are first incubated with human serum (containing pathogenic autoantibodies) and then a monoclonal capture antibody against the glycoprotein under investigation (αIIbβ3). Platelets are then lysed and the supernatant is added to a microplate precoated with an IgG against the capture antibody. Human pathogenic antibodies bound to the integrin are then detected with a peroxidase-labelled anti-human IgG.

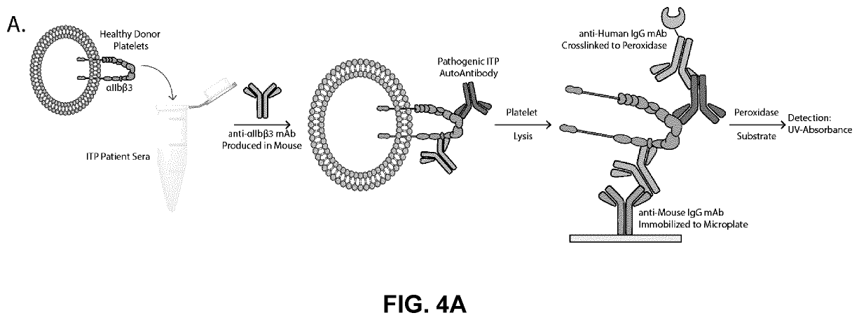

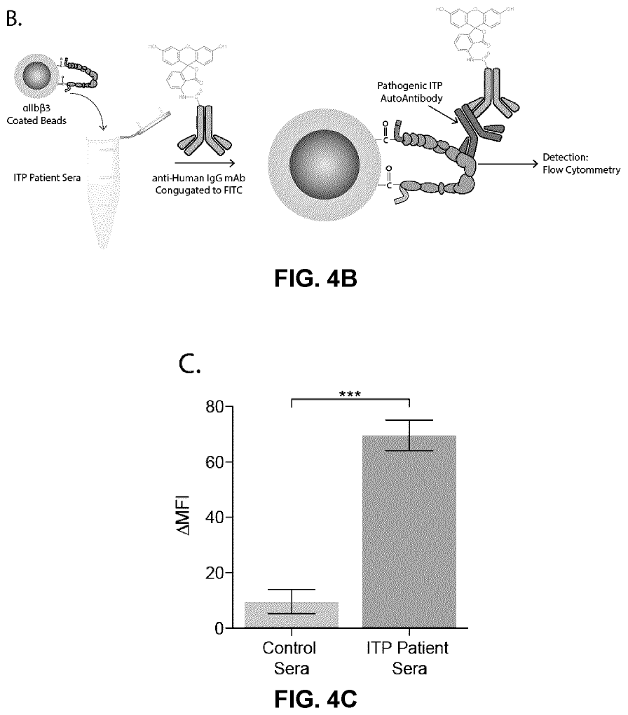

A flow cytometry assay was developed based upon the αIIbβ3 coupled beads described in Example I (FIGS. 4A to C), where both healthy control and ITP patient sera is incubated with both DETA and αIIbβ3 coupled beads. The beads are then pulled down, washed and incubated with an anti-human IgG FITC, to detect the autoantibody against αIIbβ3. The beads were then run on flow cytometry and the MFI difference between control and patient sera with each bead compared. MAIPA confirmed ITP autoantibody sera from two patients was obtained from a clinical collaborator, and analyzed using this assay (FIG. 4C), which was able to clearly detect the presence of the pathogenic anti-platelet autoantibodies.

Fibrinogen ELISA Assay. To ensure similar immobilization levels of recombinant human ectodomain αIIbβ3 between the ELISA plate and integrin coupled magnetic beads, the wells of a 96-well micro plate (Nunc MaxiSorp) were incubated with the same αIIbβ3 concentration per surface area as during the preparation of the magnetic beads, 6.6×10⁴ μg·mL⁻¹·cm⁻². Each well was coated with αIIbβ3 or control proteins (BSA and β3−/− platelet lysate) by incubation of 6 μg/μL protein in binding buffer (TRIS buffered saline with 0.05% TWEEN-20 and 1 mM each of MgCl₂, MnCl₂ and CaCl₂) at 4° C. overnight. Incubation of 3% skim milk (ED Millipore) and 2% TWEEN-20 for 1 hour at 37° C. was used for blocking. See FIGS. 5A and 5B. The concentration of αIIbβ3 per surface area of bead used when coating SAM coated beads is 6.6×10⁴ μg·mL⁻¹·cm⁻², therefore the wells of 96 well plates used were coated with the same density EDC/NHS quenching achieved by increase pH to 8.6. Bead concentration for synthesis: 6×10⁹ beads·mL⁻¹; Integrin-bead reaction concentration=250 μg·mL⁻¹; Integrin Used=6.6×10⁴ μg·mL⁻¹·cm⁻²; Well Surface Area (50 μL)=0.93 cm²; Concentration/wee for ELISA=6×10⁴ μg·mL⁻¹ (6 μL/well in 50 μL total volume); Stock [αIIbβ3]=5×10⁵ μg·mL⁻¹.

To evaluate the analytical potential of these αIIbβ3 coupled molecular probes, a flow cytometry sandwich assay was developed. In literature reports the most common method utilized to detect integrin binding interactions is ELISA, hence, the performance of the flow cytometry-based assay (FIG. 5A) was tested against traditional sandwich ELISA (FIG. 5B). ELISA was performed by immobilizing the same density of integrin (amount of integrin per surface area, bead or plate) onto the plate surface followed by blocking with 5% skim milk with 0.5% TWEEN, while the flow cytometry assay did not include a blocking step. The same primary antibody was used for both assays, however, the detection antibody for ELISA was HRP labelled and for flow cytometry was FITC labelled. The fibrinogen does response curves are depicted in the inserts of each of FIGS. 5A and 5B. The flow cytometry-based assay produced a K_{dapparant} of 0.21±0.03 μM and CLOD of 0.026±0.002 μM while ELISA produced a K_{d} of 2.2±0.4 μM and CLOD of 0.54±0.07 μM. The flow cytometry based assay produced a significant increase in performance compared to ELISA, even though a blocking step and a signal amplification-based detection strategy were employed in ELISA. It was postulated that the significant increase in performance observed is due to the SAM and the immobilization strategy which promotes the high-affinity ligand binding conformation of the integrin.

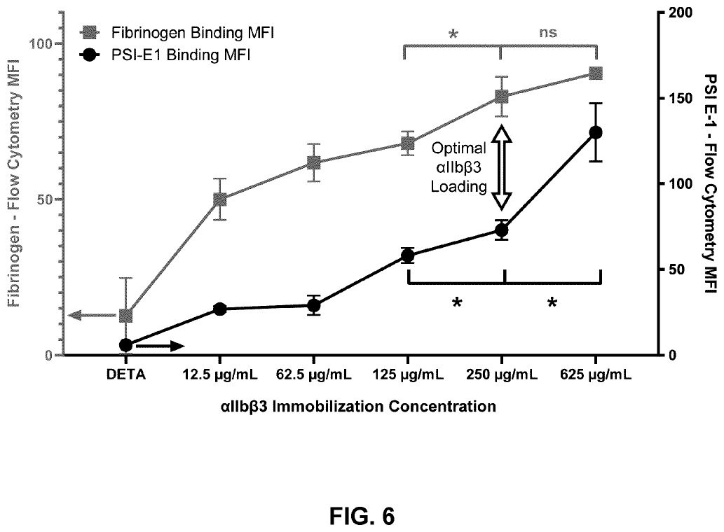

Loading optimization. At various immobilization concentrations of αIIbβ3 integrin, the resulting αIIbβ3-coupled beads were evaluated for fibrinogen or PSI-E1 binding using flow cytometry. PSI-E1 is an antibody that binds linear epitope and indicated represents the amount of integrin loaded onto the surface. While, fibrinogen is the natural ligand of αIIbβ3, and fibrinogen binding indicates the activity of the αIIbβ2-coupled beads. The optimal loading concentration was determined to be 250 μg/mL, increasing the concentration, although increases the amount of αIIbβ3 integrin on the bead surface does not increase the binding activity of the resulting αIIbβ3-coupled beads (FIG. 6).

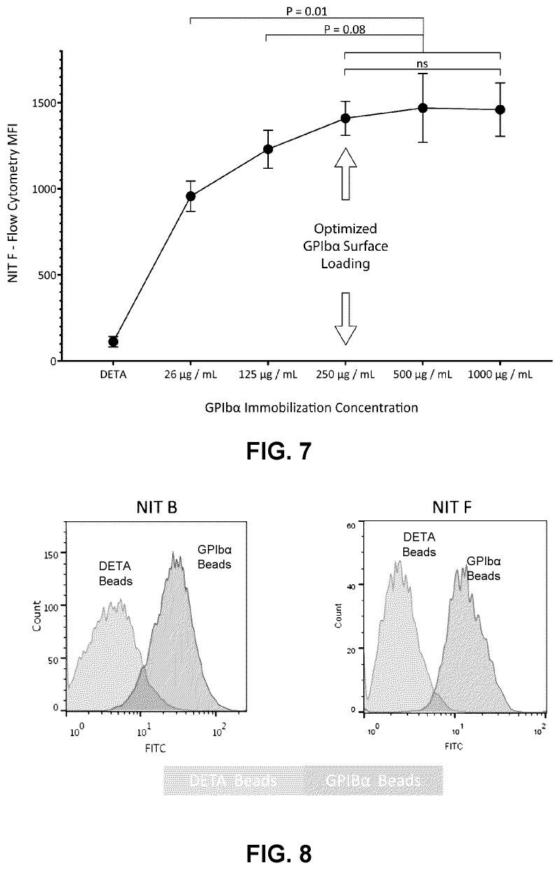

Due to the success of the assay in the proof of concept phase, the assay was compared against MAIPA in a clinical study consisting of clinical patient samples, both ITP patient and control. To expand on the scope of the assay GPIBa (to detect anti-GPIBa autoantibodies; FIGS. 7 & 8) and BSA (as another control) coated beads were added to the assay. Our flow cytometry assay (described in Example I) was much faster only requiring hours to complete instead of days. Furthermore, the flow cytometry assay produced concentration limits of detection an order of magnitude more sensitive than MAIPA for both anti-GPlba and anti-αIIbβ3 antibodies (FIG. 10), while producing much fewer false positives (i.e. positive healthy control samples). Under the conditions tested, the flow cytometry assay produced 1 false positive out of 18 control samples, while MAIPA produced 6 false positives out of the 18 control samples. Also, the flow cytometry assay detected antibodies in 22 out of 27 ITP patients while MAIPA only detected antibodies in 12 of the 27 ITP patients.

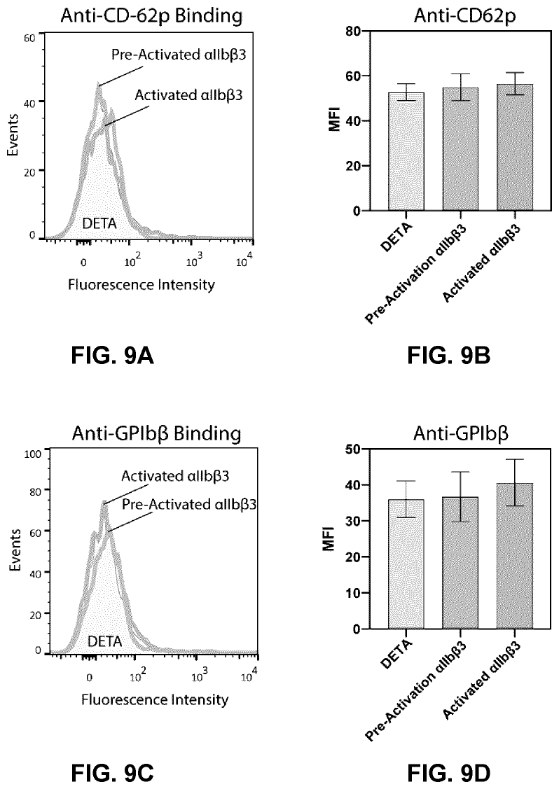

DETA, pre-activation αIIbβ3 and activated αIIbβ3 particles were also evaluated against negative control antibodies: anti-CD62p antibodies (FIGS. 9A and 9B), and anti-GPIB6 antibodies (FIGS. 9C and 9D). These negative control antibodies target other platelet surface receptors, hence no significant differences were seen between the DETA, pre-activation αIIbβ3 and activated αIIbβ3 particles.

The apparent binding affinity and limit of detection of the flow cytometry assay against the MAIPA assay were evaluated for the detection of anti-αIIbβ3 antibodies (PSI E1) and anti-GPIbα antibodies (NIT B). The respective dose-response curves are shown in FIG. 10.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Bergmeier, W., Rackebrandt, K., Schröder, W., Zirngibl, H., & Nieswandt, B. (2000). Structural and functional characterization of the mouse von Willebrand factor receptor GPIb-IX with novel monoclonal antibodies. Blood, 95(3).
Curtis, B. R., & McFarland, J. G. (2009). Detection and identification of platelet antibodies and antigens in the clinical laboratory. Immunohematology, 25(3), 125-135. http://www.ncbi.nlm.nih.gov/pubmed/20406019
Klee, G. G. (2000). Human anti-mouse antibodies. Archives of Pathology and Laboratory Medicine, 124(6), 921-923. https://doi.org/10.1007/978-3-662-49054-9_1489-1
Li, J., Van Der Wal, D. E., Zhu, G., Xu, M., Yougbare, I., Ma, L., Vadasz, B., Carrim, N., Grozovsky, R., Ruan, M., Zhu, L., Zeng, Q., Tao, L., Zhai, Z. M., Peng, J., Hou, M., Leytin, V., Freedman, J., Hoffmeister, K. M., & Ni, H. (2015). Desialylation is a mechanism of Fc-independent platelet clearance and a therapeutic target in immune thrombocytopenia. Nature Communications, 6. https://doi.org/10.1038/ncomms8737
McMillan, R., Longmire, R. L., Tavassoli, M., Armstrong, S., & Yelenosky, R. (1974). In Vitro Platelet Phagocytosis by Splenic Leukocytes in Idiopathic Thrombocytopenic Purpura. New England Journal of Medicine, 290(5), 249-251. https://doi.org/10.1056/NEJM197401312900505
Metzner, K., Bauer, J., Ponzi, H., Ujcich, A., & Curtis, B. R. (2017). Detection and identification of platelet antibodies using a sensitive multiplex assay system-platelet antibody bead array. Transfusion, 57(7), 1724-1733. https://doi.org/10.1111/trf.14122
Nieswandt, B., Bergmeier, W., Rackebrandt, K., Gessner, J. E., & Zirngibl, H. (2000). Identification of critical antigen-specific mechanisms in the development of immune thrombocytopenic purpura in mice. Blood, 96(7).
Webster, M. L., Sayeh, E., Crow, M., Chen, P., Nieswandt, B., Freedman, J., & Ni, H. (2006). Relative efficacy of intravenous immunoglobulin G in ameliorating thrombocytopenia induced by antiplatelet GPIIbIIIa versus GPIbα antibodies. Blood, 108(3), 943-946. https://doi.org/10.1182/blood-2005-06-009761
Xu, M., Li, J., Neves, M. A. D., Zhu, G., Carrim, N., Yu, R., Gupta, S., Marshall, J., Rotstein, O., Peng, J., Hou, M., Kunishima, S., Ware, J., Branch, D. R., Lazarus, A. H., Ruggeri, Z. M., Freedman, J., & Ni, H. (2018). GPIbα is required for platelet-mediated hepatic thrombopoietin generation. Blood, 132(6), 622-634. https://doi.org/10.1182/blood-2017-12-820779
Tao L et al., Platelet desialylation correlates with efficacy of first-line therapies for immune thrombocytopenia. J Hematol Oncol. 2017 Feb. 8; 10(1):46.
Peng J et al, Association of autoantibody specificity and response to intravenous immunoglobulin G therapy in immune thrombocytopenia: a multicenter cohort study. J Thromb Haemost. 2014 April; 12(4):497-504. https://doi.org/10.1111/jth.12524|.

1. A method of determining the presence of an antibody specific for a polypeptide present in blood or a blood product, the method comprising:
a) contacting (i) at least one first self-assembled polypeptide immobilized on a surface, the at least one first self-assembled polypeptide comprising a first ectodomain moiety, with (ii) a sample suspected of comprising the antibody; and
b) detecting the presence or absence of a complex between the at least one of the first self-assembled polypeptide and the antibody, wherein the presence of the complex is indicative of the presence of the antibody in the sample;
wherein the at least one first self-assembled polypeptide comprises a first chimeric polypeptide of formula (Ia) or (Ib):
NH₂-FPM-FAAL-FAT-COOH (Ia)
NH₂-FAT-FAAL-FPM-COOH (Ib)
wherein: FPM is a first polypeptide moiety derived from the polypeptide present in the blood or the blood product;
FAAL is a first optional amino acid linker;
FAT is a first amino acid tail having at least one acidic amino acid residue each having an R-group comprising a carboxyl group;
— is an amine bond;
the carboxyl group of the first chimeric polypeptide is covalently associated to a first silane linker (FSL) moiety, wherein the FSL is covalently associated with at least one first hydroxyl group of the surface; and
the at least one self-assembled polypeptide has specific affinity to the antibody.

2. The method of claim 1, wherein the sample is from a subject suspected of comprising the antibody.

3. The method of claim 1, wherein the blood product is a plasma.

4. The method of claim 3, wherein the plasma is a platelet-rich plasma or a platelet-poor plasma.

5. The method of claim 1 for diagnosing thrombocytopenia, wherein the detection of the complex is indicative of the presence of thrombocytopenia in the subject.

6. The method of claim 5, wherein the polypeptide is a polypeptide present on the surface of a platelet.

7. The method of claim 6, wherein the antibody is an allo-antibody.

8. The method of claim 7 for diagnosing alloimmune thrombocytopenia, wherein the detection of the complex is indicative of the presence of alloimmune thrombocytopenia in the subject.

9. The method of claim 7 for diagnosing fetal and neonatal alloimmune thrombocytopenia (FNAIT), wherein the detection of the complex is indicative of the presence of FNAIT in the subject and/or a gestated offspring of the subject.

10. The method of claim 7 for diagnosing post transfusion purpura (PTP), wherein the detection of the complex is indicative of the presence of PTP in the subject.

11. The method of claim 6, wherein the antibody is an auto-antibody.

12. The method of claim 11 for diagnosing drug-induced immune thrombocytopenia, wherein the detection of the complex is indicative of the presence of drug-induced immune thrombocytopenia.

13. The method of claim 11 for diagnosing autoimmune thrombocytopenia, wherein the detection of the complex is indicative of the presence of autoimmune thrombocytopenia in the subject.

14. (canceled)

15. (canceled)

16. (canceled)

17. The method of claim 1, comprising further contacting at least one second self-assembled polypeptide immobilized on the surface with the sample, the at least one second self-assembled polypeptide comprising a second ectodomain moiety, and forming a multimer with the at least one first self-assembled polypeptide;
wherein the at least one second self-assembled polypeptide comprises a second chimeric polypeptide non-covalently associated with the first chimeric polypeptide, the second chimeric polypeptide having formula (IIa) or (IIb):
NH₂-SPM-SAAL-SAT-COOH (IIa)
NH₂-SAT-SAAL-SPM-COOH (IIb)
wherein SPM is a second polypeptide moiety derived from the peptide present in the plasma or a fragment thereof;
SAAL is an optional second amino acid linker;
SAT is a second amino acid tail having at least one acidic amino acid residue each having an R-group comprising a carboxyl group; and
— is an amine bond;
wherein the carboxyl group of the second chimeric polypeptide is covalently associated to a second silane linker (SSL) moiety, wherein the SSL is covalently associated with at least one second hydroxyl group of the surface; and
wherein the FAT is non-covalently associated with the SAT.

18. The method of claim 17, wherein the FPM and the SPM are the same, and the at least one first self-assembled polypeptide forms a homomultimer with the at least one second self-assembled polypeptide.

19. The method of claim 18, wherein the FPM and the SPM are different, and the at least one first self-assembled polypeptide forms a heteromultimer with the at least one second self-assembled polypeptide.

20. The method of claim 17, wherein the surface has the FAAL and/or SAAL.

21. The method of claim 17, wherein:
the FAT is at least one and up to 50 amino acid residues in length, and has a pl of about 10; and
the SAT is at least three and up to 50 amino acid residues in length, and has a pl of about 4.

22. The method of claim 21, wherein:
the FAT has an amino acid sequence of SEQ ID NO: 4 or functional variants or fragments thereof; and
the SAT has an amino acid sequence of SEQ ID NO: 9 or functional variants or fragments thereof.

23. The method of claim 19, wherein:
the first chimeric polypeptide comprises a αIIb polypeptide, and the FPM has an amino acid sequence of SEQ ID NO: 2 or functional variants or fragments thereof; and
the second chimeric polypeptide comprises a β3 polypeptide, and the SPM has an amino acid sequence of SEQ ID NO: 7 or functional variants or fragments thereof.

24. (canceled)

25. The method of claim 1, wherein the at least one first self-assembled polypeptide is an activated receptor protein comprising a GPIbα polypeptide, and the FPM has an amino acid sequence of SEQ ID NO: 11 or functional variants or fragments thereof.

26. (canceled)

27. The method of claim 1, wherein the at least one first self-assembled polypeptide is an activated surface protein comprising a αIIb polypeptide, and the FPM has an amino acid sequence of SEQ ID NO: 2 or functional variants or fragments thereof.

28. The method of claim 1, wherein the at least one first self-assembled polypeptide is an activated surface protein comprising a β3 polypeptide, and the FPM has an amino acid sequence of SEQ ID NO: 7 or functional variants or fragments thereof.

29. (canceled)

30. (canceled)

31. The method of claim 1, wherein the sample is a blood sample.

32. The method of claim 1, comprising detecting the complex by flow cytometry or an enzyme-linked immunosorbent assay.

33. A surface for determining the presence of an antibody specific for a polypeptide present in blood or a blood product as described in claim 1, comprising the at least one first self-assembled polypeptide and the at least one second self-assembled polypeptide, the at least one first self-assembled polypeptide forming a multimer with the at least one second self-assembled polypeptide.

34. (canceled)

35. The surface of claim 33, wherein:
the FPM and the SPM are the same, and the at least one first self-assembled polypeptide forms a homomultimer with the at least one second self-assembled polypeptide; or
the FPM and the SPM are different, and the at least one first self-assembled polypeptide forms a heteromultimer with the at least one second self-assembled polypeptide.

36. (canceled)

37. The surface of claim 33, comprising a spherical surface or a planar surface.

38. (canceled)

39. (canceled)

40. (canceled)

41. A process of immobilizing at least one first self-assembled polypeptide to a surface for diagnosing thrombocytopenia, the surface having at least one first hydroxyl group covalently associated with a first silane linker moiety, the at least one first self-assembled polypeptide comprising a first chimeric polypeptide, the process comprising:
obtaining the first chimeric polypeptide as defined in claim 1; and
adding the first chimeric polypeptide to the surface in a solvent under suitable conditions for first chimeric polypeptide to covalently bond to the surface via the first silane linker moiety.

42. The process of claim 41 further comprises immobilizing at least one second self-assembled polypeptide to the surface, the surfacing further having at least one second hydroxyl group covalently associated with a second silane linker moiety, the at least one second self-assembled polypeptide forming a multimer with the at least one first self-assembled polypeptide, the at least one second self-assembled polypeptide comprising a second chimeric polypeptide, the process further comprising:
obtaining the second chimeric polypeptide as defined in claim 17; and
adding the second chimeric polypeptides to the surface in a solvent under suitable conditions for the second chimeric polypeptide to covalently bond to the surface via the second silane linker moieties respectively.

43. The process of claim 42, wherein:
the FPM and the SPM are the same, and the at least one first self-assembled polypeptide forms a homomultimer with the at least one second self-assembled polypeptide, or
the FPM and the SPM are different, and the at least one first self-assembled polypeptide forms a heteromultimer with the at least one second self-assembled polypeptide.

44. (canceled)

45. (canceled)

46. (canceled)

47. The process of claim 42, further comprising coating the surface with the first and/or second silane linker moieties by reacting with the hydroxyl groups.

48. The process of claim 42, further comprising obtaining the first chimeric polypeptide and/or the second chimeric polypeptide from recombinant expression in a recombinant host cell.

49. (canceled)

50. (canceled)

51. (canceled)

52. A kit for determining the presence of an antibody specific for a peptide present in blood or a blood product, the kit comprising (i) a first chimeric polypeptide claim 1, wherein the first chimeric polypeptide is capable of binding to an antibody to the first polypeptide moiety and (ii) a surface for covalently associating the first chimeric polypeptide, wherein the surface has first hydroxyl groups covalently associated with a first silane linker moiety.

53. The kit of claim 52, further comprising a second chimeric polypeptide as defined in claim 17, wherein:
the first and the second chimeric polypeptide are capable of forming an heteromultimer and wherein the surface further comprises second hydroxyl groups covalently associated with a second silane linker moiety; or
the first and the second chimeric polypeptide are capable of forming an homomultimer and wherein the surface further comprises second hydroxyl groups covalently associated with a second silane linker moiety.

54. (canceled)

55. (canceled)

56. (canceled)

57. The kit of claim 52, wherein the surface is a microsphere silica bead.

58. A method of treating thrombocytopenia in a subject, the method comprising:
a) detecting the expression of an antibody specific for a polypeptide in the blood or a blood product with the method of claim 1, the surface of claim 33, or the kit of claim 52 in a sample obtained from a subject suspected of comprising the antibody; and
b) administering a treatment to the subject having been determined to have the antibody specific for the polypeptide in the blood or blood product.

59. The method of claim 58 for treating alloimmune thrombocytopenia, or fetal and neonatal alloimmune thrombocytopenia (FNAIT).

60. (canceled)

61. The method of claim 59, wherein the treatment comprises administering intravenous immunoglobulin (IVIG), a steroid and/or serial intrauterine platelet transfusions (IUPT).

62. The method of claim 61 for treating autoimmune thrombocytopenia.

63. The method of claim 62 for treating drug induced immune thrombocytopenia.

64. The method of claim 62 for treating thrombotic thrombocytopenic purpura (TTP) or immune thrombocytopenic purpura (ITP).

65. (canceled)

66. (canceled)

67. The method of claim 64, wherein the antibody is an anti-GPlba autoantibody or an anti-αIIbβ3 autoantibody.

68. (canceled)

69. (canceled)

70. The method of claim 67, wherein the treatment comprises one or more of immunosuppressive agent administration, immunomodulatory agent administration, splenectomy, corticosteroid administration, intravenous immunoglobulin G (IVIG) administration, or anti-RhD therapy.

71. (canceled)
